(19) United States
(12) Patent Application Publication
(10) Pub. No.: US 2021/0318397 A1
(43) Pub. Date: Oct. 14, 2021

(54) MRI SURGICAL SYSTEMS INCLUDING MRI-COMPATIBLE SURGICAL CANNULAS FOR TRANSFERRING A SUBSTANCE TO AND/OR FROM A PATIENT

(71) Applicant: ClearPoint Neuro, Inc., Irvine, CA (US)

(72) Inventor: Peter G. Piferi, Orange, CA (US)

(21) Appl. No.: 17/021,773

(22) Filed: Sep. 15, 2020

Related U.S. Application Data
(60) Provisional application No. 63/006,929, filed on Apr. 8, 2020.

(51) Int. Cl. G01R 33/28; G01R 33/56

(52) U.S. Cl. CPC G01R 33/285; G01R 33/281; G01R 33/5601

(57) ABSTRACT

A cannula for transferring a substance to and/or from a patient includes a rigid, tubular support sleeve, an inner sleeve, a transfer tube, and a conformal polymeric sleeve. The support sleeve defines a support sleeve lumen extending from a first opening at a proximal end of the support sleeve to a second opening at a distal end of the support sleeve. The support sleeve includes a rigid, MRI-compatible material. The inner sleeve is disposed in the support sleeve lumen and extends beyond the distal end of the support sleeve to a distal end of the inner sleeve. The inner sleeve defines an inner sleeve lumen. The transfer tube is disposed in the inner sleeve lumen and extends to or beyond the distal end of the inner sleeve to a distal end of the transfer tube. The transfer tube defines a transfer tube lumen terminating at an opening at the distal end of the transfer tube. The conformal polymeric sleeve surrounds at least a portion of the support sleeve and at least a portion of the inner sleeve. The conformal polymeric sleeve includes a transitional section extending from the distal end of the support sleeve and over the inner sleeve in a direction toward the distal end of the inner sleeve. The transitional section tapers inwardly in the direction toward the distal end of the inner sleeve.

### RELATED APPLICATION(S)

The present application claims the benefit of and priority from U.S. Provisional Patent Application No. 63/006,929, filed Apr. 8, 2020, the disclosure of which is incorporated herein by reference in its entirety.

### FIELD OF THE INVENTION

The present invention relates to MRI-guided diagnostic or interventional systems that may be particularly suitable for placement/localization of therapies in the body.

### BACKGROUND OF THE INVENTION

Various therapeutic and diagnostic procedures require that a substance be delivered (e.g., dispensed or infused) into a prescribed region of a patient, such as into a target deep brain location in the patient's brain, using a delivery cannula. It is often important or critical that the substance be delivered with high accuracy to the target region in the patient and without undue trauma to the patient.

### SUMMARY OF THE INVENTION

According to some embodiments, a cannula for transferring a substance to and/or from a patient includes a rigid, tubular support sleeve, a transfer tube, and a conformal polymeric sleeve. The support sleeve defines a support sleeve lumen extending from a first opening at a proximal end of the support sleeve to a second opening at a distal end of the support sleeve. The support sleeve comprises a rigid, MRI-compatible material. The transfer tube is disposed in the support sleeve lumen and extends beyond the distal end of the support sleeve to a distal end of the transfer tube. The transfer tube defines a transfer tube lumen terminating at an opening at the distal end of the transfer tube. The conformal polymeric sleeve surrounds at least a portion of the support sleeve and at least a portion of the transfer tube. The conformal polymeric sleeve includes a transitional section extending from the distal end of the support sleeve and over the transfer tube in a direction toward the distal end of the transfer tube. The transitional section tapers inwardly in the direction toward the distal end of the transfer tube.

In some embodiments, the transitional section is frustoconical.

According to some embodiments, the transitional section extends from the distal end of the support sleeve toward the distal end of the transfer tube an axial distance in the range of from about 0.1 mm to 5 mm.

In some embodiments, the support sleeve comprises a ceramic material.

In some embodiments, the conformal polymeric sleeve is a polymeric shrink tubing.

In some embodiments, the transfer tube is formed of fused silica.

According to some embodiments, the transfer tube is adhered by adhesive to an inner surface of the support sleeve.

In some embodiments, the conformal polymeric sleeve defines an annular void between an outer surface of the transfer tube and the transitional section of the conformal polymeric sleeve adjacent the distal end of the support sleeve, and the cannula includes an adhesive at least partially filling the annular void.

In some embodiments, the conformal polymeric sleeve extends to the distal end of the transfer tube.

In some embodiments, the conformal polymeric sleeve has a distal end located axially between the distal end of the support sleeve and the distal end of the transfer tube.

According to some embodiments, the cannula includes an inner sleeve disposed in the support sleeve lumen and extending beyond the distal end of the support sleeve to a distal end of the inner sleeve. The inner sleeve defines an inner sleeve lumen. The transfer tube is disposed in the inner sleeve lumen and extends to or beyond the distal end of the inner sleeve to the distal end of the transfer tube. The conformal polymeric sleeve surrounds at least a portion of the inner sleeve. The transitional section extends from the distal end of the support sleeve and over the inner sleeve in a direction toward the distal end of the inner sleeve. The transitional section tapers inwardly in the direction toward the distal end of the inner sleeve.

In some embodiments, the conformal polymeric sleeve defines an annular void between an outer surface of the inner sleeve and the transitional section of the conformal polymeric sleeve adjacent the distal end of the support sleeve, and the cannula includes an adhesive at least partially filling the annular void.

In some embodiments, the conformal polymeric sleeve extends to the distal end of the inner sleeve.

In some embodiments, the conformal polymeric sleeve covers a terminal end face of the inner sleeve at the distal end of the inner sleeve.

In some embodiments, the transfer tube extends axially beyond the inner sleeve in the direction of the distal end of the transfer tube.

According to some embodiments, the distal end of the inner sleeve is coterminous with the distal end of the transfer tube.

In some embodiments, the conformal polymeric sleeve extends to the distal end of the inner sleeve.

In some embodiments, the conformal polymeric sleeve has a distal end located axially between the distal end of the support sleeve and the distal end of the inner sleeve.

According to some embodiments, the transfer tube extends beyond the distal end of the inner sleeve. An exterior surface of the cannula has at least first, second and third co-axially disposed segments, an outer diameter of the second segment being greater than an outer diameter of the first segment, and an outer diameter of the third segment being greater than the outer diameter of the second segment. The second segment extends between and adjoins each of the first and third segments. The first segment extends from the distal end of the transfer tube to the distal end of the inner sleeve. The second segment extends from the distal end of the inner sleeve to a distal end of the transitional section of the conformal polymeric sleeve. The third segment extends from a proximal end of the transitional section toward the proximal end of the support sleeve. A terminal end face of the inner sleeve forms a sharp step about the transfer tube between the first segment and the second segment, the terminal end face of the inner sleeve projecting radially outwardly beyond the outer diameter of the first segment. The transitional section defines a second step between the second segment and the third segment, the second step having a smooth rise from the outer diameter of the second segment to the outer diameter of the third segment.

According to some embodiments, the inner sleeve is adhered with adhesive to an inner surface of the tubular support sleeve.

According to some embodiments, the inner sleeve is adhered with adhesive to an outer surface of the transfer tube.

According to some embodiments, a method of transferring a substance to and/or from a patient includes providing a cannula including: a rigid, tubular support sleeve defining a support sleeve lumen extending from a first opening at a proximal end of the support sleeve to a second opening at a distal end of the support sleeve, wherein the support sleeve comprises a rigid, Mill-compatible material; a transfer tube disposed in the support sleeve lumen and extending beyond the distal end of the support sleeve to a distal end of the transfer tube, the transfer tube defining a transfer tube lumen terminating at an opening at the distal end of the transfer tube; and a conformal polymeric sleeve surrounding at least a portion of the support sleeve and at least a portion of the transfer tube. The conformal polymeric sleeve includes a transitional section extending from the distal end of the support sleeve and over the transfer tube in a direction toward the distal end of the transfer tube. The transitional section tapers inwardly in the direction toward the distal end of the transfer tube. The method further includes: inserting the cannula into a selected region in the patient; and transferring the substance to or from the selected region through the transfer tube lumen.

In some embodiments, the method includes: partially withdrawing the cannula from a patient tissue in the selected region, thereby forming a channel in the patient tissue; and delivering stem cells through the cannula into the channel.

In some embodiments, the selected region is the brain.

According to some embodiments, a cannula for transferring a substance to and/or from a patient includes a rigid, tubular support sleeve, an inner sleeve, a transfer tube, and a conformal polymeric sleeve. The support sleeve defines a support sleeve lumen extending from a first opening at a proximal end of the support sleeve to a second opening at a distal end of the support sleeve. The support sleeve comprises a rigid, MM-compatible material. The inner sleeve is disposed in the support sleeve lumen and extending beyond the distal end of the support sleeve to a distal end of the inner sleeve. The inner sleeve defines an inner sleeve lumen. The transfer tube is disposed in the inner sleeve lumen and extending to or beyond a distal end of the inner sleeve to a distal end of the transfer tube. The transfer tube defines a transfer tube lumen terminating at an opening at the distal end of the transfer tube. The conformal polymeric sleeve surrounds at least a portion of the support sleeve and at least a portion of the inner sleeve. The distal end of the inner sleeve is coterminous with the distal end of the transfer tube.

In some embodiments, the conformal polymeric sleeve extends to the distal end of the inner sleeve.

In some embodiments, the conformal polymeric sleeve has a distal end located axially between the distal end of the support sleeve and the distal end of the inner sleeve.

The support sleeve may comprise a ceramic material.

According to some embodiments, the conformal polymeric sleeve is a polymeric shrink tubing.

In some embodiments, the transfer tube is formed of fused silica.

According to some embodiments, a method of transferring a substance to and/or from a patient includes providing a cannula including: a rigid, tubular support sleeve defining a support sleeve lumen extending from a first opening at a proximal end of the support sleeve to a second opening at a distal end of the support sleeve, wherein the support sleeve comprises a rigid, MRI-compatible material; an inner sleeve disposed in the support sleeve lumen and extending beyond the distal end of the support sleeve to a distal end of the inner sleeve, the inner sleeve defining an inner sleeve lumen; a transfer tube disposed in the inner sleeve lumen and extending to or beyond a distal end of the inner sleeve to a distal end of the transfer tube, the transfer tube defining a transfer tube lumen terminating at an opening at the distal end of the transfer tube; and a conformal polymeric sleeve surrounding at least a portion of the support sleeve and at least a portion of the inner sleeve; wherein the distal end of the inner sleeve is coterminous with the distal end of the transfer tube. The method further includes: inserting the cannula into a selected region in the patient; and transferring the substance to or from the selected region through the transfer tube lumen.

In some embodiments, the method includes: partially withdrawing the cannula from a patient tissue in the selected region, thereby forming a channel in the patient tissue; and delivering stem cells through the cannula into the channel.

According to some embodiments, the selected region is the brain.

According to some embodiments, a cannula for transferring a substance to and/or from a patient includes a rigid, tubular support sleeve, a transfer tube, and a conformal polymeric sleeve. The support sleeve defines a support sleeve lumen extending from a first opening at a proximal end of the support sleeve to a second opening at a distal end of the support sleeve. The support sleeve comprises a rigid, MRI-compatible material. The transfer tube is disposed in the support sleeve lumen and extending beyond the distal end of the support sleeve to a distal end of the transfer tube. The transfer tube defines a transfer tube lumen terminating at an opening at the distal end of the transfer tube. The conformal polymeric sleeve surrounds at least a portion of the support sleeve and at least a portion of the transfer tube. The transfer tube is adhered by adhesive to an inner surface of the support sleeve.

In some embodiments, the conformal polymeric sleeve extends to the distal end of the transfer tube.

In some embodiments, the conformal polymeric sleeve has a distal end located axially between the distal end of the support sleeve and the distal end of the transfer tube.

According to some embodiments, the support sleeve comprises a ceramic material.

According to some embodiments, the conformal polymeric sleeve is a polymeric shrink tubing.

In some embodiments, the transfer tube is formed of fused silica.

According to some embodiments, a method of transferring a substance to and/or from a patient includes providing a cannula including: a rigid, tubular support sleeve defining a support sleeve lumen extending from a first opening at a proximal end of the support sleeve to a second opening at a distal end of the support sleeve, wherein the support sleeve comprises a rigid, MRI-compatible material; a transfer tube disposed in the support sleeve lumen and extending beyond the distal end of the support sleeve to a distal end of the transfer tube, the transfer tube defining a transfer tube lumen terminating at an opening at the distal end of the transfer tube; and a conformal polymeric sleeve surrounding at least a portion of the support sleeve and at least a portion of the transfer tube; wherein the transfer tube is adhered by adhesive to an inner surface of the support sleeve. The method further includes: inserting the cannula into a selected region in the patient; and transferring the substance to or from the selected region through the transfer tube lumen.

In some embodiments, the method includes: partially withdrawing the cannula from a patient tissue in the selected region, thereby forming a channel in the patient tissue; and delivering stem cells through the cannula into the channel.

According to some embodiments, the selected region is the brain.

### BRIEF DESCRIPTION OF THE DRAWINGS

FIG. 1 is a schematic illustration of a MM-guided surgical system according to some embodiments of the present invention.

FIG. 2 is a side perspective view of a trajectory guide forming a part of the MRI-guided surgical system of FIG. 1 mounted on a patient.

FIG. 3 is a side perspective view of a part of the MM-guided surgical system of FIG. 1 mounted on the patient.

FIG. 4 is an enlarged, perspective view of the trajectory guide of FIG. 2.

FIG. 5 is a sectional view of the trajectory guide of FIG. 2 with a surgical cannula forming a part of the MM-guided surgical system according to embodiments of the present invention.

FIGS. 6A-6C and 7A-7B are schematic illustrations of exemplary screen shots of displays of a User Interface provided to a user to facilitate navigation and/or assessment steps of an MRI-guided substance transfer (e.g., delivery) procedure.

FIG. 8A is a side view of a surgical cannula according to further embodiments of the present invention.

FIG. 8B is a cross-sectional view of the surgical cannula of FIG. 8A taken along the line 8B-8B of FIG. 8A.

FIG. 8C is an enlarged, cross-sectional view of Detail 8C of FIG. 8B.

FIG. 8D is an enlarged, cross-sectional view of Detail 8D of FIG. 8B.

FIG. 8E is an enlarged, cross-sectional view of Detail 8E of FIG. 8B.

FIG. 8F is a further enlarged, cross-sectional view of Detail 8E of FIG. 8B.

FIG. 9A is a fragmentary, cross-sectional view of a surgical cannula according to further embodiments of the present invention.

FIG. 9B is an enlarged, fragmentary, cross-sectional view of a midsection of the surgical cannula FIG. 9A.

FIG. 9C is an enlarged, fragmentary, cross-sectional view of a tip section of the surgical cannula FIG. 9A.

FIG. 10A is a fragmentary, cross-sectional view of a surgical cannula according to further embodiments of the present invention.

FIG. 10B is an enlarged, fragmentary, cross-sectional view of a midsection of the surgical cannula FIG. 10A.

FIG. 10C is an enlarged, fragmentary, cross-sectional view of a tip section of the surgical cannula FIG. 10A.

FIG. 11A is a fragmentary, cross-sectional view of a surgical cannula according to further embodiments of the present invention.

FIG. 11B is an enlarged, fragmentary, cross-sectional view of a midsection of the surgical cannula FIG. 11A.

FIG. 11C is an enlarged, fragmentary, cross-sectional view of a tip section of the surgical cannula FIG. 11A.

FIG. 12A is a fragmentary, cross-sectional view of a surgical cannula according to further embodiments of the present invention.

FIG. 12B is an enlarged, fragmentary, cross-sectional view of a midsection of the surgical cannula FIG. 12A.

FIG. 13 is a data processing system according to some embodiments of the present invention.

FIG. 14 is an exemplary screen shot of a display of a User Interface during an MRI-guided substance delivery procedure.

FIGS. 15A and 15B are exemplary screen shots of a display of a User Interface during an MRI-guided substance delivery procedure and captured at a first time and a second, subsequent time, respectively.

### DETAILED DESCRIPTION OF EMBODIMENTS OF THE INVENTION

The present invention will now be described more fully hereinafter with reference to the accompanying drawings, in which embodiments of the invention are shown. This invention may, however, be embodied in many different forms and should not be construed as limited to the embodiments set forth herein; rather, these embodiments are provided so that this disclosure will be thorough and complete, and will fully convey the scope of the invention to those skilled in the art. Like numbers refer to like elements throughout. It will be appreciated that although discussed with respect to a certain embodiment, features or operation of one embodiment can apply to others.

In the drawings, the thickness of lines, layers, features, components and/or regions may be exaggerated for clarity and broken lines (such as those shown in circuit of flow diagrams) illustrate optional features or operations, unless specified otherwise. In addition, the sequence of operations (or steps) is not limited to the order presented in the claims unless specifically indicated otherwise.

The terminology used herein is for the purpose of describing particular embodiments only and is not intended to be limiting of the invention. As used herein, the singular forms “a”, “an” and “the” are intended to include the plural forms as well, unless the context clearly indicates otherwise. It will be further understood that the terms “comprises” and/or “comprising,” when used in this specification, specify the presence of stated features, steps, operations, elements, and/or components, but do not preclude the presence or addition of one or more other features, steps, operations, elements, components, and/or groups thereof. As used herein, the term “and/or” includes any and all combinations of one or more of the associated listed items.

Unless otherwise defined, all terms (including technical and scientific terms) used herein have the same meaning as commonly understood by one of ordinary skill in the art to which this invention belongs. It will be further understood that terms, such as those defined in commonly used dictionaries, should be interpreted as having a meaning that is consistent with their meaning in the context of the specification and relevant art and should not be interpreted in an idealized or overly formal sense unless expressly so defined herein. Well-known functions or constructions may not be described in detail for brevity and/or clarity.

It will be understood that when a feature, such as a layer, region or substrate, is referred to as being “on” another feature or element, it can be directly on the other element or intervening elements may also be present. In contrast, when an element is referred to as being “directly on” another feature or element, there are no intervening elements present. It will also be understood that, when a feature or element is referred to as being “connected” or “coupled” to another feature or element, it can be directly connected to the other element or intervening elements may be present. In contrast, when a feature or element is referred to as being “directly connected” or “directly coupled” to another element, there are no intervening elements present. Although described or shown with respect to one embodiment, the features so described or shown can apply to other embodiments.

The term “electroanatomical visualization” refers to a visualization or map of the anatomical structure, e.g., brain, typically a volumetric, 3-D map or 4-D map, that illustrates or shows electrical activity of tissue correlated to anatomical and/or coordinate spatial position. The visualization can be in color and color-coded to provide an easy to understand map or image with different measures or gradients of activity in different colors and/or intensities. The term “color-coded” means that certain features, electrical activity or other output are shown with defined colors of different color and/or intensity to visually accentuate different tissue, different and similar electrical activity or potential in tissue and/or to show abnormalities or lesions in tissue versus normal or non-lesion tissue. In some embodiments, the systems can be configured to allow a clinician to increase or decrease the intensity or change a color of certain tissue types or electrical outputs, e.g., in high-contrast color and/or intensity, darker opacity or the like.

The actual visualization can be shown on a screen or display so that the map and/or anatomical or tool structure is in a flat 2-D view and/or in 2-D what appears to be 3-D volumetric images with data representing features or electrical output with different visual characteristics such as with differing intensity, opacity, color, texture and the like. For example, the 3-D image of the lung can be generated to illustrate differences in barrier thickness using color or opacity differences over the image volume. Thus, the term “3-D” in relation to images does not require actual 3-D viewability (such as with 3-D glasses), just a 3-D appearance, typically on a display. The 3-D images comprise multiple 2-D slices. The 3-D images can be volume renderings well known to those of skill in the art and/or a series of 2-D slices, which can be visually paged through. A 4-D map illustrates time-dependent activity, such as electrical activity or blood flow movement.

The surgical systems may be configured to operate based on known physical characteristics of one or more surgical tools, which may include a surgical (e.g., delivery) cannula, such that the hardware is a point of interface for the circuit or software. The systems can communicate with databases that define dimensions, configurations or shapes and spacing of components on the tool(s). The defined physical data can be obtained from a CAD model of a tool. The physical characteristics can include dimensions or other physical features or attributes and may also include relative changes in position of certain components or features upon a change in position of a tool or portion thereof. The defined physical characteristics can be electronically (programmatically) accessible by the system or known a priori and electronically stored locally or remotely and used to automatically calculate certain information and/or to segment image data. That is, tool data from the known dimensions and configuration of the tool model can be used to segment image data and/or correlate a position and orientation of a tool and/or provide trajectory adjustment guidelines or error estimates, warnings of improper trajectories and the like. For example, the system can include defined structural and/or operational details/data for one or more of a delivery cannula, a grid for marking a burr hole location and/or a trajectory guide. The system can use this data to allow a user to adjust an intrabrain path for placing a diagnostic or therapy device. Such can be input, transposed, and/or overlayed in a visualization of the tool on one or more displays along with patient structure or otherwise used, such as, for example, to project the information onto a patient's anatomical structure or determine certain operational parameters including which image volume (scan planes) to use to obtain MRI image data that will include select portions of the targeting cannula or surgical cannula. As such, at least some of the generated visualizations are not merely an MRI image of the patient during a procedure.

The visualizations are rendered visualizations that can combine multiple sources of data to provide visualizations of spatially encoded tool position and orientation with anatomical structure and can be used to provide position adjustment data output so that a clinician can obtain a desired trajectory path, thereby providing a smart-adjustment system without requiring undue “guess” work on what adjustments to make to obtain the desired trajectory.

The term “animation” refers to a sequence or series of images shown in succession, typically in relatively quick succession, such as in about 1-50 frames per second. The term “frame” refers to a single visualization or static image. The term “animation frame” refers to one image frame of the different images in the sequence of images.

The term “ACPC coordinate space” refers to a right-handed coordinate system defined by anterior and posterior commissures (AC, PC) and Mid-Sagittal plane points, with positive directions corresponding to a patient's anatomical Right, Anterior and Head directions with origin at the mid-commissure point.

The term “grid” refers to a pattern of crossed lines or shapes used as a reference for locating points or small spaces, e.g., a series of rows and intersecting columns, such as horizontal rows and vertical columns (but orientations other than vertical and horizontal can also be used). The grid can include associated visual indicia such as alphabetical markings (e.g., A-Z and the like) for rows and numbers for columns (e.g., 1-10) or the reverse. Other marking indicia may also be used. The grid can be provided as a flexible patch that can be releasably attached to the skull of a patient. For additional description of suitable grid devices, see co-pending, co-assigned U.S. patent application Ser. No. 12/236,621 (U.S. Published Patent Application No. US 2009/0177077 A1), the disclosure of which is incorporated herein by reference.

The term “fiducial marker” refers to a marker that can be electronically identified using image recognition and/or electronic interrogation of MRI image data. The fiducial marker can be provided in any suitable manner, such as, but not limited to, a geometric shape of a portion of the tool, a component on or in the tool, a coating or fluid-filled component or feature (or combinations of different types of fiducial markers) that makes the fiducial marker(s) MRI-visible with sufficient signal intensity (brightness) or generates a “void” or dark space for identifying location and/or orientation information for the tool and/or components thereof in space.

The term “Mill scanner” refers to a magnetic resonance imaging and/or NMR spectroscopy system. As is well known, Mill scanners include a low field strength magnet (typically between about 0.1T to about 0.5T), a medium field strength magnet, or a high-field strength super-conducting magnet, an RF pulse excitation system, and a gradient field system. MRI scanners are well known to those of skill in the art. Examples of commercially available clinical Mill scanners include, for example, those provided by General Electric Medical Systems, Siemens, Philips, Varian, Bruker, Marconi, Hitachi and Toshiba. The MRI systems can be any suitable magnetic field strength, such as, for example, about 1.5T or about 3.0T, and may include other high-magnetic field systems between about 2.0T-10.0T.

The term “RF safe” means that the lead or probe is configured to safely operate when exposed to RF signals, particularly RF signals associated with MRI systems, without inducing unplanned current that inadvertently unduly heats local tissue or interferes with the planned therapy.

The term “MRI visible” means that the device is visible, directly or indirectly, in an MRI image. The visibility may be indicated by the increased SNR of the MRI signal proximate the device.

The term “MRI compatible” means that the so-called component(s) is suitable for use in an MM environment and as such is typically made of a non-ferromagnetic MM compatible material(s) suitable to reside and/or operate in or proximate a conventional medical high magnetic field environment. The “MM compatible” component or device is “MR safe” when used in the MM environment and has been demonstrated to neither significantly affect the quality of the diagnostic information nor have its operations affected by the MR system at the intended use position in an MR system. These components or devices may meet the standards defined by ASTM F2503-05. See, American Society for Testing and Materials (ASTM) International, Designation: F2503-05. Standard Practice for Marking Medical Devices and Other Items for Safety in the Magnetic Resonance Environment. ASTM International, West Conshohocken, Pa., 2005.

The term “near real time” refers to both low latency and high frame rate. Latency is generally measured as the time from when an event occurs to display of the event (total processing time). For tracking, the frame rate can range from between about 100 fps to the imaging frame rate. In some embodiments, the tracking is updated at the imaging frame rate. For near ‘real-time’ imaging, the frame rate is typically between about 1 fps to about 20 fps, and in some embodiments, between about 3 fps to about 7 fps. The low latency required to be considered “near real time” is generally less than or equal to about 1 second. In some embodiments, the latency for tracking information is about 0.01 s, and typically between about 0.25-0.5 s when interleaved with imaging data. Thus, with respect to tracking, visualizations with the location, orientation and/or configuration of a known intrabody device can be updated with low latency between about 1 fps to about 100 fps. With respect to imaging, visualizations using near real time MR image data can be presented with a low latency, typically within between about 0.01 ms to less than about 1 second, and with a frame rate that is typically between about 1-20 fps. Together, the system can use the tracking signal and image signal data to dynamically present anatomy and one or more intrabody devices in the visualization in near real-time. In some embodiments, the tracking signal data is obtained and the associated spatial coordinates are determined while the MR image data is obtained and the resultant visualization(s) with the intrabody device (e.g., surgical cannula) and the near RT MR image(s) are generated.

The term “automatically” means that the operation can be substantially, and typically entirely, carried out without human or manual input, and is typically programmatically directed or carried out. The term “electronically” includes both wireless and wired connections between components. The term “programmatically” means under the direction of a computer program that communicates with electronic circuits and other hardware and/or software.

The term “surgical cannula” refers to an intrabody cannula used to transfer a substance to and/or from a target intrabody location.

Embodiments of the invention may be particularly suitable for use with human patients but may also be used with any animal or other mammalian subject.

Embodiments of the present invention may take the form of an entirely software embodiment or an embodiment combining software and hardware aspects, all generally referred to herein as a “circuit” or “module.” In some embodiments, the circuits include both software and hardware and the software is configured to work with specific hardware with known physical attributes and/or configurations. Furthermore, the present invention may take the form of a computer program product on a computer-usable storage medium having computer-usable program code embodied in the medium. Any suitable computer readable medium may be utilized including hard disks, CD-ROMs, optical storage devices, a transmission media such as those supporting the Internet or an intranet, or other storage devices.

Computer program code for carrying out operations of the present invention may be written in an object oriented programming language such as Java®, Smalltalk or C++. However, the computer program code for carrying out operations of the present invention may also be written in conventional procedural programming languages, such as the “C” programming language. The program code may execute entirely on the user's computer, partly on the user's computer, as a stand-alone software package, partly on the user's computer and partly on another computer, local and/or remote or entirely on the other local or remote computer. In the latter scenario, the other local or remote computer may be connected to the user's computer through a local area network (LAN) or a wide area network (WAN), or the connection may be made to an external computer (for example, through the Internet using an Internet Service Provider).

Embodiments are described in part below with reference to flowchart illustrations and/or block diagrams of methods, apparatus (systems) and computer program products according to embodiments of the invention. It will be understood that each block of the flowchart illustrations and/or block diagrams, and combinations of blocks in the flowchart illustrations and/or block diagrams, can be implemented by computer program instructions. These computer program instructions may be provided to a processor of a general purpose computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks.

These computer program instructions may also be stored in a computer-readable memory that can direct a computer or other programmable data processing apparatus to function in a particular manner, such that the instructions stored in the computer-readable memory produce an article of manufacture including instruction means which implement the function/act specified in the flowchart and/or block diagram block or blocks.

The computer program instructions may also be loaded onto a computer or other programmable data processing apparatus to cause a series of operational steps to be performed on the computer or other programmable apparatus to produce a computer implemented process such that the instructions which execute on the computer or other programmable apparatus provide steps for implementing the functions/acts specified in the flowchart and/or block diagram block or blocks.

The flowcharts and block diagrams of certain of the figures herein illustrate exemplary architecture, functionality, and operation of possible implementations of embodiments of the present invention. In this regard, each block in the flow charts or block diagrams represents a module, segment, or portion of code, which comprises one or more executable instructions for implementing the specified logical function(s). It should also be noted that in some alternative implementations, the functions noted in the blocks may occur out of the order noted in the figures. For example, two blocks shown in succession may in fact be executed substantially concurrently or the blocks may sometimes be executed in the reverse order or two or more blocks may be combined, depending upon the functionality involved.

Some embodiments of the present invention are directed to MM-guided systems that can generate substantially real time (e.g., near real-time) patient-specific visualizations of the patient and one or more surgical tools, including an MRI-compatible intrabody surgical cannula (e.g., delivery cannula) and the delivery distribution, location, pattern, etc., in logical space and provide feedback to a clinician to improve the speed and/or reliability of an intrabody infusion or delivery of a substance to a target within the body through the delivery cannula. The delivery cannula has at least one lumen and at least one exit port configured to direct a flow of the substance through the lumen and the exit port to the target.

Some embodiments of the present invention are directed to MM-guided systems that can generate substantially real time patient-specific visualizations of the patient and a distribution of a substance delivered to a target within the patient through an MRI-compatible delivery cannula in logical space and provide feedback to a clinician to improve the speed and/or reliability of an intrabody infusion or delivery of the substance. These systems can show a dynamic dispersion and/or infusion pattern of the substance delivered or dispensed into the patient. MRI can be effectively used to monitor the efficacy and/or delivery of the substance from the delivery cannula.

The visualizations can be based (in-part) on predefined data of the delivery cannula which can define a point of interface for the system (e.g., software) based on predefined characteristics of the delivery cannula, e.g., dimensions, shape or configuration and/or known rotational, translational and/or other functional and/or dynamic behavior of the delivery cannula (e.g., flow rate, nozzle angle). The visualizations can include patient function data (e.g., fMRI data, electrical activity, active regions of a brain during a defined stimulation, fiber tracks, and the like).

The system can be configured to interrogate and segment image data to locate fiducial markers in the image (e.g., an increased higher intensity pixel/voxel region and/or void created in the MRI image by the presence of the delivery cannula in the patient's tissue) and generate successive visualizations of the patient's anatomical structure and the delivery cannula using MRI image data and a priori data of the delivery cannula to provide (substantially real-time) visualizations of the distribution of the substance in the patient.

Some embodiments of the present invention can provide visualizations to allow more precise control, delivery, and/or feedback of an infusion or delivery therapy so that the therapy or delivery cannula associated therewith can be more precisely placed and/or so that the cannula or delivery can be adjusted to provide the desired distribution in tissue, or to confirm proper delivery and allow near real-time visualization of the procedure.

Some embodiments of the present invention are directed to MRI-compatible intrabody delivery cannulas that may be used to precisely deliver any suitable and desired substance (e.g., cellular, biological, and/or drug therapeutics) to the desired anatomy target. The delivery cannulas can be used in and the systems can be configured to guide and/or place the delivery cannula in any desired internal region of the body of the patient, but may be particularly suitable for neurosurgeries and delivery of a substance to a target area or region within the brain. The delivery cannulas and systems can be used for gene and/or stem-cell based therapy delivery or other neural therapy delivery and allow user-defined custom targets in the brain or to other locations. Cannulas, systems and methods of the invention may be used to treat patients by delivery of cellular/biological therapeutics into the desired anatomy to modify their cellular function. The cells (e.g., stem cells) may improve function.

The target region may be any suitable region or area within the patient body. According to some embodiments, the target region is a STN anatomical region, which may be identified and located with reference to standard anatomical landmarks. According to some embodiments, the target area is a deep brain tumor or other undesirable tissue mass.

According to some embodiments, the target is intrathecal. The intrathecal target may be in the brain or spinal cord.

The substance delivered to the target region through the delivery cannula may be any suitable and desired substance. According to some embodiments, the substance is a liquid or slurry. In the case of a tumor, the substance may be a chemotherapeutic (cytotoxic) fluid. In some embodiments, the substance can include certain types of advantageous cells that act as vaccines or other medicaments (for example, antigen presenting cells such as dentritic cells). The dentritic cells may be pulsed with one or more antigens and/or with RNA encoding one or more antigen. Exemplary antigens are tumor-specific or pathogen-specific antigens. Examples of tumor-specific antigens include, but are not limited to, antigens from tumors such as renal cell tumors, melanoma, leukemia, myeloma, breast cancer, prostate cancer, ovarian cancer, lung cancer and bladder cancer. Examples of pathogen-specific antigens include, but are not limited to, antigens specific for HIV or HCV. In some embodiments, the substance may comprise radioactive material such as radioactive seeds. Substances delivered to a target area may include, but are not limited to, the following as shown in Table 1:

TABLE 1

| DRUG (generic name) | DISORDER(S) |
|---|---|
| caprylidene | Alzheimer's disease |
| donepezil | Alzheimer's disease |
| galantamine | Alzheimer's disease |
| memantine | Alzheimer's disease |
| Tacrine | Alzheimer's disease |
| vitamin E | Alzheimer's disease |
| ergoloid mesylates | Alzheimer's disease |
| riluzole | Amyotrophic lateral sclerosis |
| metoprolol | Benign essential tremors |
| primidone | Benign essential tremors |
| propanolol | Benign essential tremors |
| gabapentin | Benign essential tremors & Epilepsy |
| nadolol | Benign essential tremors & Parkinson's disease |
| zonisamide | Benign essential tremors & Parkinson's disease |
| carmustine | Brain tumor |
| lomustine | Brain tumor |
| methotrexate | Brain tumor |
| cisplatin | Brain tumor & Neuroblastoma |
| ioversol | Cerebral arteriography |
| mannitol | Cerebral Edema |
| dexamethasone | Cerebral Edema & Neurosarcoidosis |
| baclofen | Cerebral spasticity |
| ticlopidine | Cerebral thrombosis/embolism |
| isoxsuprine | Cerebrovascular insufficiency |
| cefotaxime | CNS infection & Meningitis |
| acyclovir | Encephalitis |
| foscarnet | Encephalitis |
| ganciclovir | Encephalitis |
| interferon alpha-2a | Encephalitis |
| carbamazepine | Epilepsy |
| clonazepam | Epilepsy |
| diazepam | Epilepsy |
| divalproex sodium | Epilepsy |
| ethosuximide | Epilepsy |
| ethotoin | Epilepsy |
| felbamate | Epilepsy |
| fosphenytoin | Epilepsy |
| levetiracetam | Epilepsy |
| mephobarbital | Epilepsy |
| paramethadione | Epilepsy |
| phenytoin | Epilepsy |
| trimethadione | Epilepsy |
| pregabalin | Epilepsy & Neuralgia |
| immune globulin intravenous | Guillain-Barre Syndrome |
| interferon beta-1b | Guillain-Barre Syndrome & Multiple sclerosis |
| azathioprine | Guillain-Barre Syndrome & Multiple sclerosis & |
| | Neurosarcoidosis |
| risperidone | Head injury |
| tetrabenazine | Huntington's disease |
| acetazolamide | Hydrocephalus & Epilepsy |
| alteplase | Ischemic stroke |
| clopidogrel | Ischemic stroke |
| nimodipine | Ischemic stroke & Subarachnoid hemorrhage |
| Aspirin | Ischemic stroke & Thromboembolic stroke |
| amikacin | Encaphalitis |
| ampicillin | Encaphalitis |
| ampicillin/sulbactam | Encaphalitis |
| ceftazidime | Encaphalitis |
| ceftizoxime | Encaphalitis |
| cefuroxime | Encaphalitis |
| chloramphenicol | Encaphalitis |
| cilastatin/imipenem | Encaphalitis |
| gentamicin | Encaphalitis |
| meropenem | Encaphalitis |
| metronidazole | Encaphalitis |
| nafcillin | Encaphalitis |
| oxacillin | Encaphalitis |
| piperacillin | Encaphalitis |
| rifampin | Encaphalitis |
| sulfamethoxazole/trimethoprim | Encaphalitis |
| tobramycin | Encaphalitis |
| triamcinolone | Encaphalitis |
| vancomycin | Encaphalitis |
| ceftriaxone | Encaphalitis & Neurosyphilis |
| pennicillin | Encaphalitis & Neurosyphilis |
| corticotropin | Multiple sclerosis |
| dalfampridine | Multiple sclerosis |
| glatiramer | Multiple sclerosis |
| mitoxantrone | Multiple sclerosis |
| natalizumab | Multiple sclerosis |
| modafinil | Multiple sclerosis |
| cyclophosphamide | Multiple sclerosis & Brain tumor & Neuroblastoma |
| interferon beta-1a | Multiple sclerosis & Neuritis |
| prednisolone | Multiple sclerosis & Neurosarcoidosis |
| prednisone | Multiple sclerosis & Neurosarcoidosis |
| amantadine | Multiple sclerosis & Parkinson's disease |
| methylprednisolone | Neuralgia |
| desvenlafaxine | Neuralgia |
| nortriptyline | Neuralgia |
| doxorubicin | Neuroblastoma |
| vincristine | Neuroblastoma |
| albendazole | Neurocystecercosis |
| chloroquine phosphate | Neurosarcoidosis |
| hydroxychloroquine | Neurosarcoidosis |
| infliximab | Neurosarcoidosis |
| pentoxyfilline | Neurosarcoidosis |
| thalidomide | Neurosarcoidosis |
| apomorphine | Parkinson's disease |
| belladonna | Parkinson's disease |
| benztropine | Parkinson's disease |
| biperiden | Parkinson's disease |
| bromocriptine | Parkinson's disease |
| carbidopa | Parkinson's disease |
| carbidopa/entacapone/levodopa | Parkinson's disease |
| carbidopa/levodopa | Parkinson's disease |
| entacapone | Parkinson's disease |
| levodopa | Parkinson's disease |
| pergolide mesylate | Parkinson's disease |
| pramipexole | Parkinson's disease |
| procyclidine | Parkinson's disease |
| rasagiline | Parkinson's disease |
| ropinirole | Parkinson's disease |
| rotiotine | Parkinson's disease |
| scopolamine | Parkinson's disease |
| tolcapone | Parkinson's disease |
| trihexyphenidyl | Parkinson's disease |
| seleginline | Parkinson's disease |
| rivastigmine | Parkinson's disease & Alzheimer's disease |
| anisindione | Thromboembolic stroke |
| warfarin | Thromboembolic stroke |
| 5-hydroxytryptophan | Depression & Anxiety & ADHD |
| duloxetine | Depression & Anxiety & Bipolar disorder |
| escitalopram | Depression & Anxiety & Bipolar disorder |
| venlafaxine | Depression & Anxiety & Bipolar disorder & Autism & |
| | Social anxiety disorder |
| desvenlafaxine | Depression & Anxiety & PTSD & ADHD |
| paroxetine | Depression & Anxiety & PTSD & Social anxiety disorder |
| fluoxetine/olanzapine | Depression & Bipolar disorder |
| l-methylfolate | Depression & BPD |
| amitriptyline | Depression & PTSD |
| sertraline | Depression & PTSD & Bipolar disorder & Social anxiety |
| | disorder |
| fluvoxamine | Depression & PTSD & Social anxiety disorder |
| olanzapine | Depression & Schizophrenia & Bipolar disorder |
| paliperidone | Depression & Schizophrenia & Bipolar disorder |
| aripiprazole | Depression & Schizophrenia & Bipolar disorder & Autism |
| quetiapine | Depression & Schizophrenia & PTSD & BPD & Bipolar |
| | disorder |
| risperidone | Depression & Schizophrenia & PTSD & BPD & Bipolar |
| | disorder & Autism |
| amisulpride | Depression & Social anxiety disorder |
| chlorpromazine | Psychosis |
| droperidol | Psychosis |
| fluphenazine | Psychosis |
| periciazine | Psychosis |
| perphenazine | Psychosis |
| thiothixene | Psychosis |
| triflupromazine | Psychosis |
| haloperidol | Psychosis & Dementia |
| prazosin | PTSD |
| clozapine | Schizophrenia |
| flupenthixol | Schizophrenia |
| iloperidone | Schizophrenia |
| loxapine | Schizophrenia |
| mesoridazine | Schizophrenia |
| promazine | Schizophrenia |
| reserpine | Schizophrenia |
| thioridazein | Schizophrenia |
| zuclopenthixol | Schizophrenia |
| asenapine | Schizophrenia & Bipolar disorder |
| levomepromazine | Schizophrenia & Bipolar disorder |
| ziprasidone | Schizophrenia & Bipolar disorder |
| molindone | Schizophrenia & Psychosis |
| pimozide | Schizophrenia & Psychosis |
| thioridazine | Schizophrenia & Psychosis |
| Cytarabine | Chemotherapy, hematological malignancies |

According to some embodiments, the surgical cannula is used to remove or withdraw a substance therethrough from the target area. According to some embodiments, the surgical cannula is used to remove cerebral spinal fluid from the patient.

Embodiments of the present invention may include steps, features, aspects, components, procedures and/or systems as disclosed in U.S. Published Patent Application No. 2009/0171184, and PCT Published Patent Application No. WO 2011/130107 A2, the disclosures of which are incorporated herein by reference.

According to some embodiments, the systems are configured to provide a substantially automated or semi-automated and relatively easy-to-use MRI-guided system with defined workflow steps and interactive visualizations. In particular embodiments, the systems define and present workflow with discrete steps for finding target and entry point(s), guiding the alignment of the targeting cannula to a planned trajectory, monitoring the insertion of the delivery cannula, and adjusting the (X-Y) position in cases where the placement needs to be corrected. During steps where specific MR scans are used, the circuit or computer module can display data for scan plane center and angulation to be entered at the console. The workstation/circuit can passively or actively communicate with the MR scanner. The system can also be configured to use functional patient data (e.g., fiber tracks, fMRI and the like) to help plan or refine a target surgical site and/or access path.

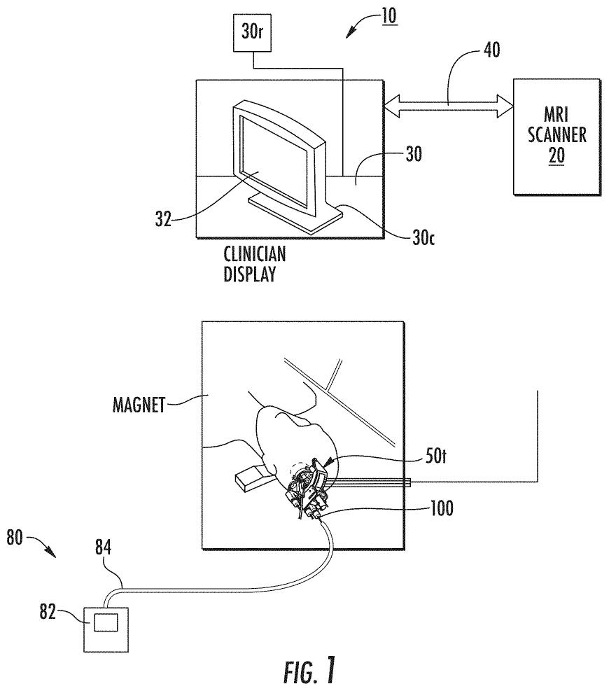

Embodiments of the present invention will now be described in further detail below with reference to the figures. FIG. 1 illustrates an MRI-guided interventional system 10 with an MRI scanner 20, a clinician workstation 30 with at least one circuit 30c, at least one display 32, an MRI compatible trajectory guide 50t, a depth stop 70 (FIG. 5), and a fluid substance delivery system 80. The fluid substance delivery system includes an MRI-compatible intrabody surgical or delivery cannula 100, an infusion or delivery pump 82 and connecting tubing 84. The system 10 can be configured to render or generate real time visualizations of the target anatomical space using MRI image data and predefined data of at least one surgical tool to segment the image data and place the trajectory guide 50t and the cannula 100 in the rendered visualization in the correct orientation and position in 3D space, anatomically registered to a patient. The trajectory guide 50t and the cannula 100 can include or cooperate with tracking, monitoring and/or interventional components.

The tools of the system 10, including the cannula 100, can be provided as a sterile kit (typically as single-use disposable hardware) or in other groups or sub-groups or even individually, typically provided in suitable sterile packaging. The tools can also include a marking grid (e.g., as disclosed in U.S. Published Patent Application No. 2009/0177077 and/or U.S. Published Patent Application No. 2009/0171184). Certain components of the kit may be replaced or omitted depending on the desired procedure. Certain components can be provided in duplicate for bilateral procedures.

With reference to FIG. 5, the depth stop 70 has a generally cylindrical configuration with opposite proximal and distal ends 70a, 70b and is adapted to be removably secured within the proximal end of the tubular trajectory guide member 50t. The depth stop 70 can be attached to the cannula 100 to allow for a defined insertion depth where insertion depth control and/or locking, is desired.

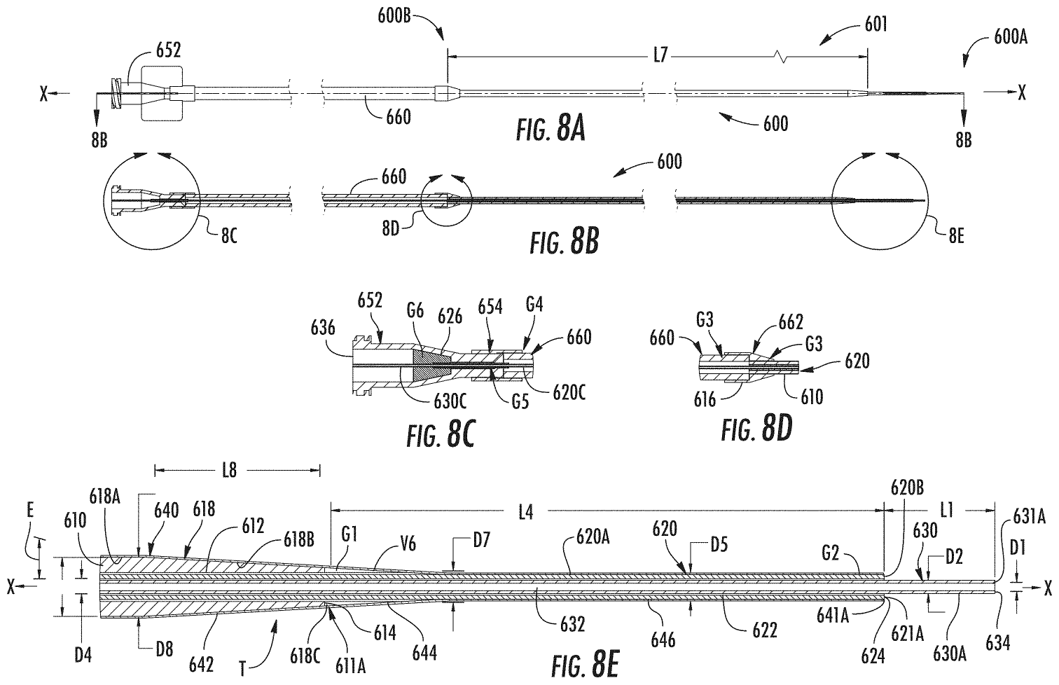

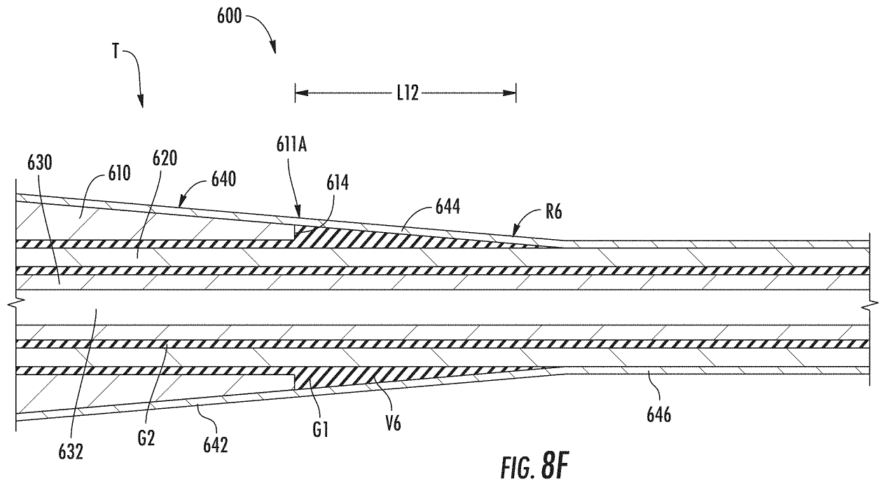

The cannula 100 can be configured to flowably introduce and/or inject a desired therapy (e.g., antigen, gene therapy, chemotherapy or stem-cell or other therapy type). The cannula 100 as shown in FIG. 5 includes a cannula body 110 defining at least one longitudinally extending lumen 112, a first or inlet port 114 and at least one second or exit port 116. The cannula 100 is formed of an MM-compatible, MRI-visible material such as ceramic. The cannula 100 as depicted in FIG. 5 is a relatively simple embodiment and further embodiments including additional functionality will be disclosed hereinbelow with reference to FIGS. 8A-12B. These further embodiments of cannula may be used in the same manner as described with regard to the cannula 100.

The lumen 112 can be fluidly connected to the pump 82 by the tubing 84. The tubing 84 may be flexible tubing. According to some embodiments, the tubing 84 is PVC tubing. According to some embodiments, the tubing 84 is silicone tubing. In some embodiments, the cannula can be self-loading and not require tubing and/or a pump. See, e.g. co-pending U.S. Provisional Patent Application Ser. No. 62/950,521 (Attorney Docket 9450-129PR), the contents of which are hereby incorporated by reference as if recited in full herein.

According to some embodiments and with reference to FIG. 3, the pump 82 includes a reservoir 83 of the substance to be delivered and a pump mechanism 81. The pump mechanism 81 is selectively operable to supply the substance to the lumen 112 under a controlled pressure. According to some embodiments, a syringe (e.g., a hand syringe) is used in place of the pump 82.

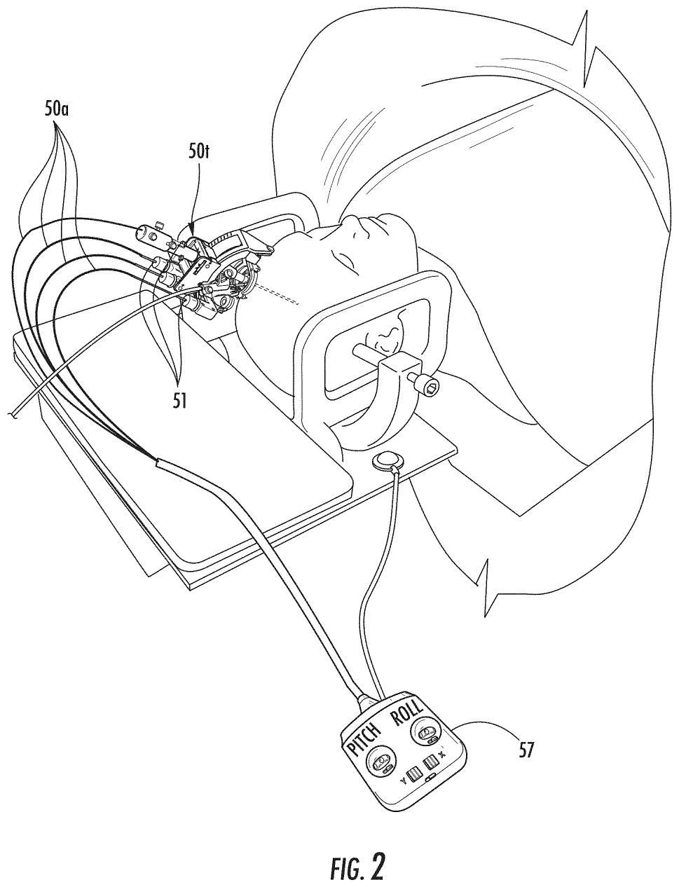

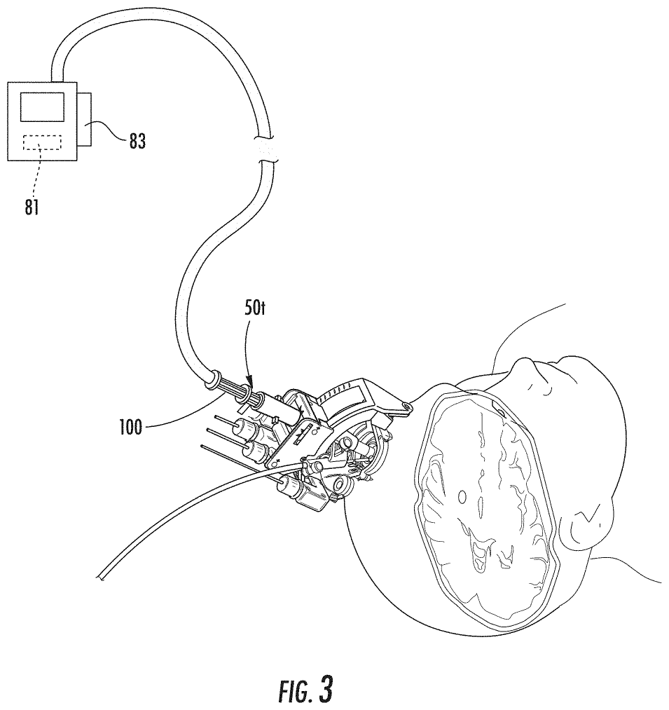

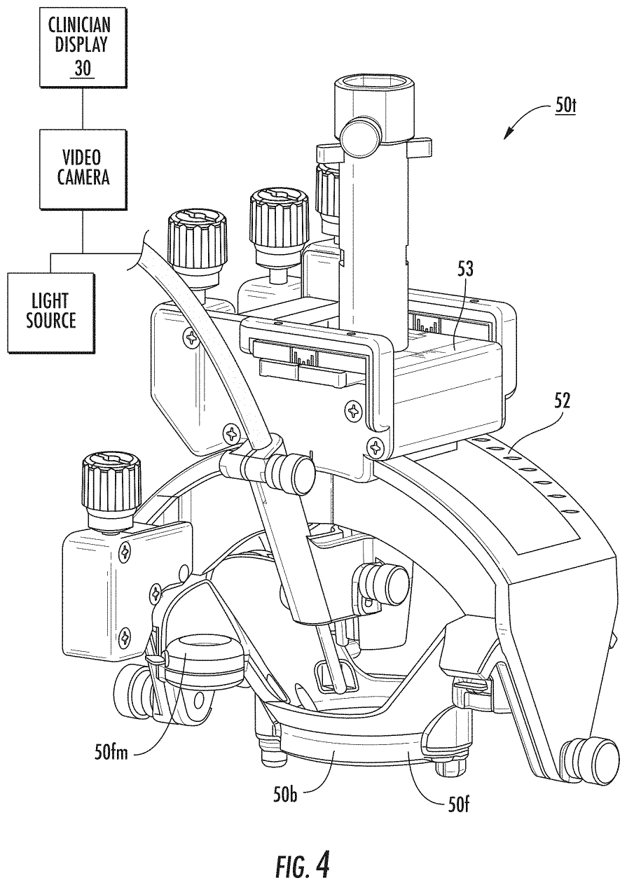

An exemplary trajectory guide 50t is illustrated in FIGS. 1-3 in position on a patient. As shown, the trajectory guide 50t (FIG. 4) includes a guide frame 50f, a targeting cannula 60 and trajectory guide actuators 51 having respective actuator cables 50a (FIG. 2) (providing X-Y adjustment and pitch and roll adjustment) in communication with a trajectory adjustment controller 57. The frame 50f can include a control arc 52 that cooperates with a platform 53 to provide pitch and roll adjustments. The platform 53 can allow for X-Y adjustments of the trajectory. The trajectory guide 50t may include a plurality of MM-visible frame fiducial markers 50fm around a base 50b thereof. For additional discussion of suitable trajectory guides, see, U.S. Published Patent Application No. 2009/0112084 (Attorney Docket No. 9450-341P), the contents of which are hereby incorporated by reference as if recited in full herein.

As shown in FIG. 5, the targeting cannula 60 includes an open center lumen or through passage 61 along the axis of the targeting cannula 60. The distal end portion of the targeting cannula 60 can include a fiducial marker 60m (typically including a fluid-filled component 65), shown as a substantially spherical or round (cross-section) marker shape. The proximal end 60p can be configured with a fluid filled channel 68 concentric with the passage 61 that can define a cylindrical fiducial marker. Other fiducial marker types can be used. The cannula 100 can be slidably introduced and/or withdrawn through the lumen or passage 61.

An MRI scanner interface 40 (FIG. 1) may be used to allow communication between the workstation 30 and the scanner 20. The interface 40 and/or circuit 30c may be hardware, software or a combination of same. The interface 40 and/or circuit 30c may reside partially or totally in the scanner 20, partially or totally in the workstation 30, or partially or totally in a discrete device therebetween.

The MRI scanner 20 can include a console that has a “launch” application or portal for allowing communication to the circuit 30c of the workstation 30. The scanner console can acquire volumetric T1-weighted (post-contrast scan) data or other image data (e.g., high resolution image data for a specific volume) of a patient's head or other anatomy. In some embodiments, the console can push DICOM images or other suitable image data to the workstation 30 and/or circuit 30c. The workstation 30 and/or circuit 30c can be configured to passively wait for data to be sent from the MR scanner 20 and the circuit 30c/workstation 30 does not query the scanner or initiate a communication to the scanner. In other embodiments, a dynamic or active communication protocol between the circuit 30c/workstation 30 and the scanner 20 may be used to acquire image data and initiate or request particular scans and/or scan volumes. Also, in some embodiments, pre-DICOM, but reconstructed image data, can be sent to the circuit 30c/workstation 30 for processing or display. In other embodiments, pre-reconstruction image data (e.g., substantially “raw” image data) can be sent to the circuit 30c/workstation 30 for Fourier Transform and reconstruction.

Generally described, for some unilateral scenarios, the user (e.g., doctor or surgeon) will proceed through a set of discrete workflow steps to load MR image data, identify a target point, identify an entry point, verify the planned trajectory, and align the targeting cannula 60. A target point or region can also be planned or refined based on real-time functional image data of a patient. The functional image data can include, but is not limited to, images of fiber tracks, images of activity in brain regions during vocalization (e.g., reading, singing, talking), or based on physical or olfactory or sense-based stimulation, such as exposure to electrical (discomfort/shock input), heat and/or cold, light or dark, visual images, pictures or movies, chemicals, scents, taste, and sounds or the like) and/or using fMRI or other imaging techniques. The enhanced visualization may give neurosurgeons a much clearer picture of the spatial relationship of a patient's brain structures. The visualizations can serve as a trajectory guide for delivering a substance to the body (e.g., to the brain) via the surgical (intrabody) delivery cannula 100. In some embodiments, the visualizations can be generated using data collated from different types of brain-imaging methods, including conventional magnetic resonance imaging (MRI), functional MM (fMRI), diffusion-tensor imaging (DTI) and even hyperpolarized noble gas MM imaging. The MRI gives details on the anatomy, fMRI or other active stimulation-based imaging protocol can provide information on the activated areas of the brain, and DTI provides images of the network of nerve fibers connecting different brain areas. The fusion of one or all of these different images and the tool information can be used to produce a 3-D display with trajectory information that surgeons can manipulate.

Thus, a target location and trajectory can be planned, confirmed or refined based in-part on functional information of the patient. This functional information can be provided, in a user interface (UI) displayed on the display screen 32, in near real-time visualizations of the patient with the trajectory guide tools for trajectory path and target planning, e.g., visualize a patient's fiber track structures and/or functional information of a patient's brain for a surgeon's ease of reference. Knowing where susceptible or sensitive brain regions are or where critical fiber tracks are in the patient's brain, can allow a surgeon to plan a better or less-intrusive trajectory and/or allow a surgeon to more precisely reach a desired target site and/or more precisely place a device and/or deliver a planned therapy substance.

To align the targeting cannula 60, scan volumes can be defined by the system based on known dimensions of the cannula, such as a cannula length, a position of a proximal or distal marker on the cannula, and angulation and lateral (X-Y) pivot limit.

An estimated distance from the distal tip of the cannula 100 to a reference point on the guide frame 50t (FIGS. 2 and 3) or the targeting cannula 60 (e.g., the proximal end of the targeting cannula 60) can be determined and physically or visually marked on the cannula 100. The depth stop 70 can be secured about the cannula 100 at the marked location. The depth stop 70 can serve to limit the depth of insertion of the cannula 100 into the patient in a subsequent insertion step or steps.

The user can then (gradually) advance the cannula 100 and acquire images (on the display of the UI) to verify the trajectory and/or avoid functionally sensitive structure as appropriate. When the delivery cannula 100 has been advanced to the target point, high-resolution confirmation images can be obtained to verify the cannula tip location relative to the planned location. Additionally or alternatively, electrical activity can be sensed using an electrode at a tip of the cannula 100. If actual placement is not correct, the cannula 100 can be withdrawn. At that point, either the X-Y placement can be adjusted appropriately (e.g., by moving a platform or stage an amount to cause the desired adjustment) or a trajectory angulation can be re-planned and a second attempt can be made.

For some bilateral scenarios, the above steps can be repeated for both left and right sides, with the additional goal that the patient should not be moved into or out of the scanner. To satisfy that goal, trajectory planning should be completed for both sides prior to removing the patient from the scanner. Also, burring and frame attachment (the member that holds the trajectory guide to the patient's head) should be completed for both sides prior to moving the patient back into the scanner 20 to promote speed of the procedure.

The system 10 can be configured with a (hardware/software) interface that provides a network connection, e.g., a standard TCP/IP over Ethernet network connection, to provide access to MR scanner 20, such as the DICOM server. The workstation 30 can provide a DICOM C-STORE storage class provider. The scanner console can be configured to be able to push images to the workstation 30 and the workstation 30 can be configured to directly or indirectly receive DICOM MR image data pushed from an MR scanner console. Alternatively, as noted above, the system can be configured with an interface that allows for a dynamic and interactive communication with the scanner 20 and can obtain image data in other formats and stages (e.g., pre-DICOM reconstructed or raw image data).

As noted above, the system 10 is configured so that hardware, e.g., the trajectory guide 50t and/or the cannula 100, constitute a point of interface with the system (software or computer programs) because the circuit 30c is configured with predefined tool data that recognizes physical characteristics of specific tool hardware.

The system 10 may also include and implement a marking grid and/or non-uniformly spaced-apart frame fiducial markers as disclosed in U.S. patent application Ser. No. 12/236,854, published as U.S. Published Patent Application No. 2009/0171184.

In some embodiments, circuit 30c can be configured so that the program application can have distinct ordered workflow steps that are organized into logical groups based on major divisions in the clinical workflow as shown in Table 2. A user may return to previous workflow steps if desired. Subsequent workflow steps may be non-interactive if requisite steps have not been completed. The major workflow groups and steps can include the following features or steps in the general workflow steps of “start”, “plan entry”, “plan target”, “navigate”, and “refine,” ultimately leading to delivering and visualizing the therapy (i.e., delivering the substance to the target through the cannula 100) as described in Table 2.

TABLE 2
Exemplary Clinical Workflow Groups/Steps

| Group | Step | Description |
|---|---|---|
| Start | Start | Set overall procedure parameters (Optionally |
| | | confirm hardware compatibility) |
| Plan Entry | ACPC | Acquire a volume and determine AC, PC, and |
| | | MSP points |
| | Target | Define initial target point(s) for entry planning |
| | Trajectory | Explore potential trajectories to determine entry |
| | | point(s) |
| | Grid | Locate physical entry point via fiducial grid. |
| Plan Target | ACPC | With hole burred and frame attached, acquire a |
| | | volume and determine revised AC, PC, and MSP |
| | | points. |
| | Target | Acquire high-resolution slabs (e.g, T2 slabs) to |
| | | determine target positions in new volume. |
| | Trajectory | Review final planned trajectory prior to starting |
| | | procedure. |
| Navigate | Initiate | Acquire slabs to locate initial position of |
| | | cannula. |
| | Alignment | Dynamically re-acquire scan showing position of |
| | | top of cannula. With each update show projected |
| | | target position to determine when alignment is |
| | | correct. |
| | Insertion | Acquire slabs as cannula 100 is inserted into |
| | | brain. Verify that cannula 100 is following |
| | | planned trajectory. |
| Refine | Target | Acquire images with cannula 100 in place. |
| | | Review position and redefine target if necessary. |
| | Adjust XY | Dynamically re-acquire scan showing position of |
| | Offset | bottom of targeting cannula 60. With each |
| | | update show projected target position to |
| | | determine when offset is correct. |
| | Insertion | Acquire slabs as cannula 100 is inserted into |
| | | brain. Verify that cannula 100 is following |
| | | planned trajectory. |
| Delivery or | Substance | Once cannula 100 position is finalized, prompt |
| Withdrawal | Delivery | user to begin delivery or withdrawal of |
| | | substance through cannula 100. |
| | Watch Diffusion | Acquire slabs as substance is delivered into or |
| | Delivery Pattern | withdrawn from brain and display. |
| Admin | Admin | Reporting and Archive functionality |

The AC, PC and MSP locations can be identified in any suitable manner. In some embodiments, the AC-PC step can have an automatic, electronic AC, PC MSP Identification Library. The AC, PC and MSP anatomical landmarks define an AC-PC coordinate system, e.g., a Talairach-Tournoux coordination system that can be useful for surgical planning. This library can be used to automatically identify the location of the landmarks. It can be provided as a dynamic linked library that a host application can interface through a set of Application Programming Interface (API) on Microsoft Windows®. This library can receive a stack of MR brain images and fully automatically locates the AC, PC and MSP. The success rate and accuracy can be optimized, and typically it takes a few seconds for the processing. The output is returned as 3D coordinates for AC and PC, and a third point that defines the MSP. This library is purely computation and is typically UI-less. This library can fit a known brain atlas to the MR brain dataset. The utility can be available in form of a dynamic linked library that a host application can interface through a set of Application Programming Interface (API) on Microsoft Windows®. The input to this library can contain the MR brain dataset and can communicate with applications or other servers that include a brain atlas or include a brain atlas (e.g., have an integrated brain atlas). The design can be independent of any particular atlas; but one suitable atlas is the Cerefy® atlas of brain anatomy (note: typically not included in the library). The library can be configured to perform segmentation of the brain and identify certain landmarks. The atlas can then be fitted in 3D to the dataset based on piecewise affine transformation. The output can be a list of vertices of the interested structures.

In some embodiments, the mid-sagittal plane (MSP) is approximated using several extracted axial slices from the lower part of the input volume, e.g., about 15 equally spaced slices. A brightness equalization can be applied to each slice and an edge mask from each slice can be created using a Canny algorithm. A symmetry axis can be found for each edge mask and identify the actual symmetry axis based on an iterative review and ranking or scoring of tentative symmetry axes. The ranking/scoring cam be based on whether a point on the Canny mask, reflected through the symmetry axis lands on the Canny mask (if so, this axes is scored for that slice). An active appearance model (AAM) can be applied to a brain stem in a reformatted input stack with the defined MSP to identify the AC and PC points.

The MSP plane estimate can be refined as well as the AC and PC points. The MSP plane estimate can be refined using a cropped image with a small region that surrounds a portion of the brain ventricle and an edge mask using a Canny algorithm. The symmetry axis on this edge mask if found following the procedure described above. The AC and PC points are estimated as noted above using the refined MSP and brightness peaks in a small region (e.g., 6×6 mm) around the estimate are searched. The largest peak is the AC point. The PC point can be refined using the PC estimate above and the refined MSP. A Canny edge map of the MSP image can be computed. Again, a small region (e.g., about 6 mm×6 mm) can be searched for a point that lies on a Canny edge and for which the image gradient is most nearly parallel to the AC-PC direction. The point is moved about 1 mm along the AC-PC direction, towards PC. The largest intensity peak in the direction perpendicular to AC-PC is taken to be the PC point.

It will be appreciated that when the target is a tumor or ventricle to be infused or the like, the AC-PC points typically will not be used to provide guidance.

The Navigation-Insertion step may include further aspects as described in Table 3A:

TABLE 3A
Navigate - Insertion
Description

The application can provide a depth value to set on the cannula 100 prior to
insertion.
The application can prompt with scan parameters for oblique coronal and sagittal
planes aligned to the trajectory. Also for an oblique axial perpendicular to the
trajectory.
On receiving coronal or sagittal images, the application can display an overlay
graphic indicating the planned trajectory. The most recent coronal and sagittal
images can appear together in a 1 × 2 display.
On receiving a trajectory axial scan perpendicular to the trajectory, the application
can segment out the cross-sections of the cannula 100 to determine the actual path
being followed by the cannula 100.
On receiving a trajectory axial scan perpendicular to the trajectory, the application
can display two viewports containing:
the axial stack with graphic overlays showing the detected path of the cannula 100
on each image
an anatomic axial view through the target showing the planned target and the target
projected from the detected path of the cannula 100. An error value can show the
distance between the current projected target and the planned target.
If multiple trajectories have been defined for a single entry, the application can
display the trajectory that is currently aligned during insertion.

The application may provide a depth value that is the expected distance from the target to the top of the targeting cannula 60. The operator can measure the depth value distance from the distal tip of the cannula 100 and mark the proximal end point on the cannula 100 (e.g., with a sterile marker). The depth stop 70 can then be secured at the marked location and the measured insertion distance verified. The depth stop 70 is configured to limit a distance that the cannula 100 extends into the body of a patient when the depth stop is inserted within the targeting cannula 60, so that full insertion of the cannula 100 up to the depth stop will provide the desired insertion depth.

In the event that the placement is not acceptable, the user may opt to proceed to the X-Y Adjustment workflow step as described in Table 3B:

TABLE 3B
Refine - Adjust X-Y Offset
Description

The X-Y Adjustment step can display the current target and projected point as
annotations to the image data that was acquired during the Target Refinement step.
This step can prompt the user to acquire 2D images with scan plane parameters
such that the image lies perpendicular to the trajectory and through the pivot point.
On receiving a 2D image through the pivot point, the step can calculate the current
projected target.
This step can display lines from the current projected target to the revised target
that indicate the track the projected target would travel if the X and Y offset
wheels were turned independently. The lines can be colored to match colors on the
control wheels for X and Y offset respectively. A tool-tip (e.g., pop-up) can
provide text to describe the necessary action.
(For example: “Turn X-offset knob to the Left”)
This step can display an annotation indicating the location of the original planned
target.
When drawing the target and the current projection of the cannula path, the
annotations can be drawn to match the physical size of the cannula 100 diameter.

After the cannula 100 has been placed and the position has been accepted by the user, the user may proceed to the substance delivery or withdrawal step.

Again, it is noted that functional patient data can be obtained in near real-time and provided to the circuit 30c/workstation 30 on the display 32 with the visualizations of the patient anatomy to help in refining or planning a trajectory and/or target location for placement of the cannula 100.

The system 10 can provide a UI to set target points so that the trajectories through potential entry points can be investigated. The user may opt to overlay the outlines from a standard brain atlas over the patient anatomy for comparison purposes which may be provided in color with different colors for different structures. When using the brain atlas, the user may opt to show either just the target structure (STN or GPi) or all structures. In either case, a tooltip (e.g., pop-up) can help the user to identify unfamiliar structures. The user may also opt to scale and/or shift the brain atlas relative to the patient image to make a better match. To do this, the user may drag the white outline surrounding the brain atlas template. Fiber track structures and/or functional information of a patient's brain can be provided in a visually prominent manner (e.g., color coded or other visual presentation) for a surgeon's ease of reference.

The UI can display images and information that enable the user to see how well the cannula 100 is following the planned trajectory. The user may opt to scan Axial, Coronal and Sagittal slabs along the cannula 100 to visually determine the cannula 100 alignment in those planes. The user can also scan perpendicular to the cannula 100. In that case, the circuit 30c (e.g., software) can automatically identify where the cannula 100 is in the slab and it then shows a projection of the current path onto the target plane to indicate the degree and direction of error if the current path is continued. The user can perform these scans multiple times during the insertion. The automatic segmentation of the cannula 100 and the display of the projected target on the target plane provide fully-automatic support for verifying the current path. The Coronal/Sagittal views can provide the physician with a visual confirmation of the cannula 100 path that does not depend on software segmentation.

After completing the initial insertion of the cannula 100, the user (e.g., physician) may find that either the placement does not correspond sufficiently close or perfectly to the plan, or the plan was not correct. The UI can support functionality whereby the physician can withdraw the cannula 100 and use the X and Y offset adjustments to obtain a parallel trajectory to a revised target. The UI can prompt the user or otherwise acquire an image slab through the distal tip of the cannula 100. The UI can display the slab and on it the user may opt to modify the target point to a new location or accept the current position as final.

The UI can also support the user in adjusting a small X-Y offset to set the targeting cannula 60 to a trajectory parallel to the original one. The UI can provide visualization of the position of the cannula 100 tip relative to the target and with instructions on what physical adjustments to make to obtain the desired parallel trajectory (shown as “turn Y wheel to the right”) and the projected error.

After the angular and/or X-Y adjustments are made, the cannula 100 insertion is carried out in the same manner as described above.

After the cannula 100 has been inserted and had its position verified by the physician, the UI can prompt the physician to begin delivery of the substance to the target via the cannula 100. In some embodiments, a test spray of a biocompatible fluid of similar density to the target therapeutic substance (e.g., saline) may be first delivered to the target.

The physician (or other operator) then actuates the pump (or syringe) 82 to begin driving a flow of the therapeutic substance through the tubing 84 and the lumen 112 of the cannula 100. A mass flow of the substance exits the cannula 100 through the exit port 116 into the target region T or the vicinity of the target region T.

Using MM image data, the system 10 may render or generate near real time visualizations of the infused or delivered substance along with the near real time visualizations of the target anatomical space and the cannula 100 in the UI. That is, in the same or similar manner to the segmentation and visualization/display of the patient anatomy, the application can segment out the cross-sections of the delivered substance to determine the actual volume occupied by the delivered substance. Scans of scan planes proximate the distal tip of the cannula 100 or associated with target regions can be acquired. The MR image data can be obtained and the actual distribution of the delivered substance in tissue can be shown on the display. These visualizations can be dynamically rendered (e.g., in near real time) to show the dynamic dispersion and/or infusion pattern and/or path of the delivered substance. In some embodiments, an MR contrast agent or fluid can be provided in the delivered substance having an increased SNR relative to the tissue.

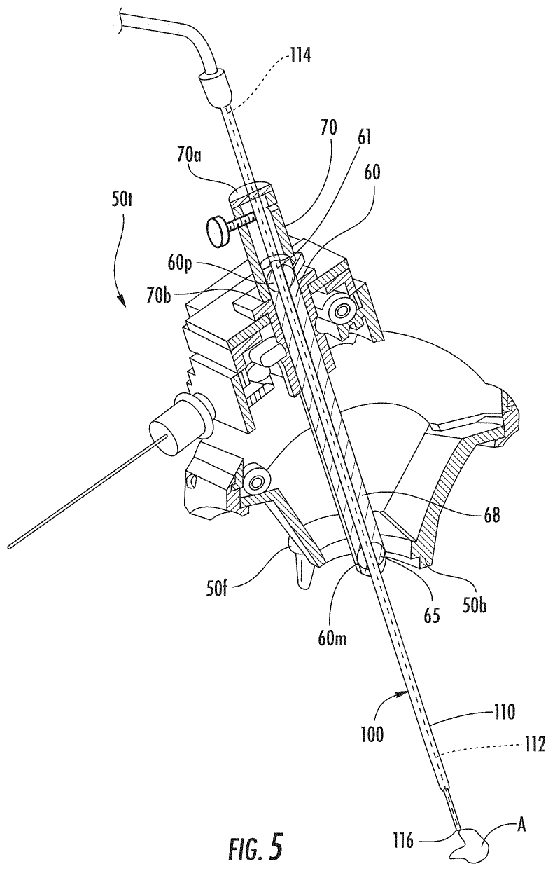

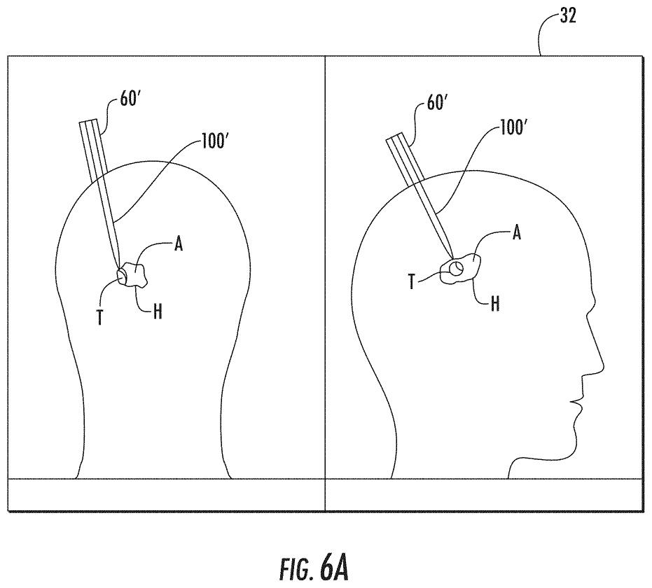

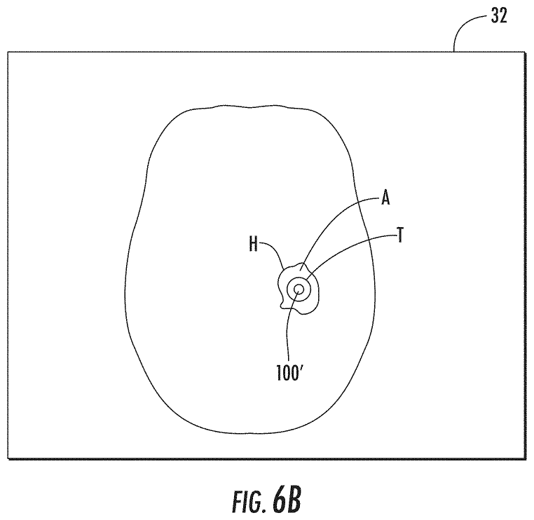

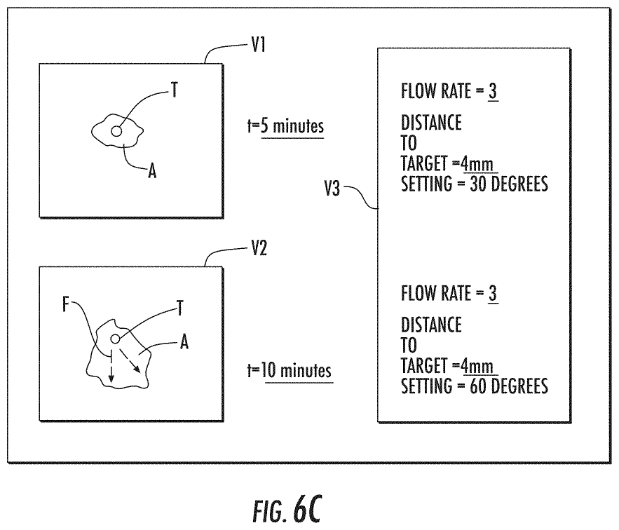

FIG. 6A illustrates a screen shot from the UI that allows the user to see the distribution of the delivered substance, which is represented in the UI by the displayed image A. This screen shot illustrates coronal and sagittal views to the target (e.g., STN). The user may opt to scan Coronal and Sagittal slabs along the cannula 100 to visually determine the distribution pattern or flow paths in those planes. The user can also scan perpendicular to the cannula 100. The cannula 100 is represented in the screen shot by an image 100′ and the targeting cannula 60 is represented in the screen shot by an image 60′. FIG. 6B illustrates an axial slab through the target T. The user can perform these scans multiple times during delivery of the substance. The automatic segmentation and display of the delivered substance, the patient anatomy and the cannula 100 provide fully-automatic support for verifying or assessing the anatomical flow paths, diffusion, dispersion, permeation and/or other distribution of the delivered substance in the patient.

The delivered substance may be visually highlighted in the display of the UI. For example, a graphical overlay or outline H may be provided in one or more of the displayed views that highlights the image of the delivered substance A. By way of further example, the image of the delivered substance A in the tissue may be provided with a contrasting coloring or shading.

As noted above, the operator can perform scans multiple times during the procedure to track or assess the delivery performance. The UI may allow the operator to display the MR image data in a manner that assists the operator in comparing the flow and/or distribution of the delivered substance over time. FIG. 6C illustrates a screen shot from the UI including a view V1 including an image A of the delivered substance at a first time (e.g., “t=5 minutes”) and a view V2 including an image A of the delivered substance A at a second, subsequent time (e.g., “t=10 minutes”). The UI may also display (e.g., in a view V3) relevant data such as the pump settings, flow rate, delivery cannula port settings, and/or nozzle angle at each of the selected times or any intervening adjustments. The UI may also display indicia F, such as graphical arrows indicating the directions of flow or dispersion, for example, between the selected times or over time. The foregoing visualization aids may assist the operator in assessing the effect of adjustments in flow rates, delivery cannula placement, or delivery cannula settings, for example.

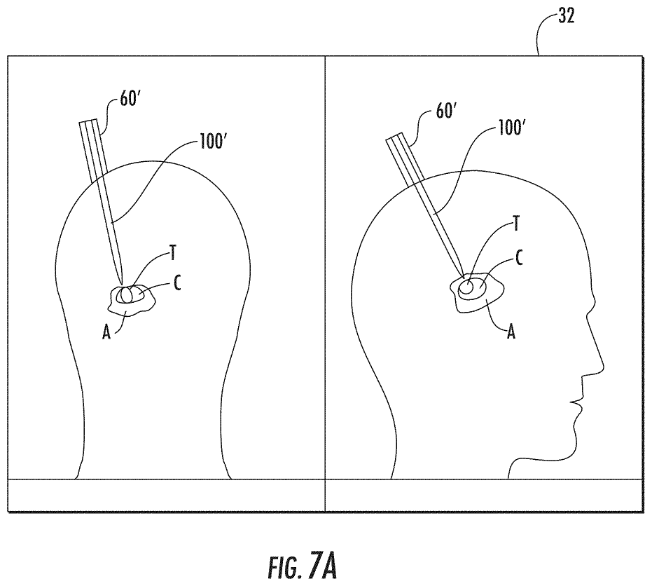

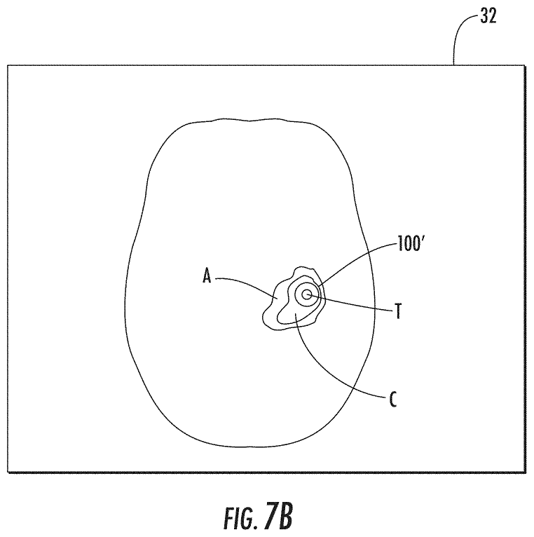

FIG. 7A illustrates a screen shot from the UI in the case where the target region is a tumor site in a patient's brain. The tumor site is segmented and displayed as a part of the patient anatomy and the image C of the tumor may be highlighted, enhanced, contrasted or otherwise augmented to enable the operator to more easily discern the tumor image C in the displayed image. The UI enables the user to see the distribution of the delivered or infused substance with respect to the tumor site. This screen shot illustrates coronal and sagittal views through the tumor. The user may opt to scan Coronal and Sagittal slabs along the cannula 100 to visually determine the distribution pattern or flow paths in those planes. The user can also scan perpendicular to the cannula 100. FIG. 7B illustrates an axial slab through the tumor. The user can perform these scans multiple times during delivery of the substance. The automatic segmentation and display of the delivered substance, the patient anatomy (including the tumor) and the cannula 100 provide fully-automatic support for verifying or assessing the anatomical flow paths, diffusion, dispersion and distribution of the delivered substance with respect to the tumor site and the remainder of the patient anatomy.

The operator can use the feedback from the UI to assess, re-plan and/or modify the infusion or delivery procedure.

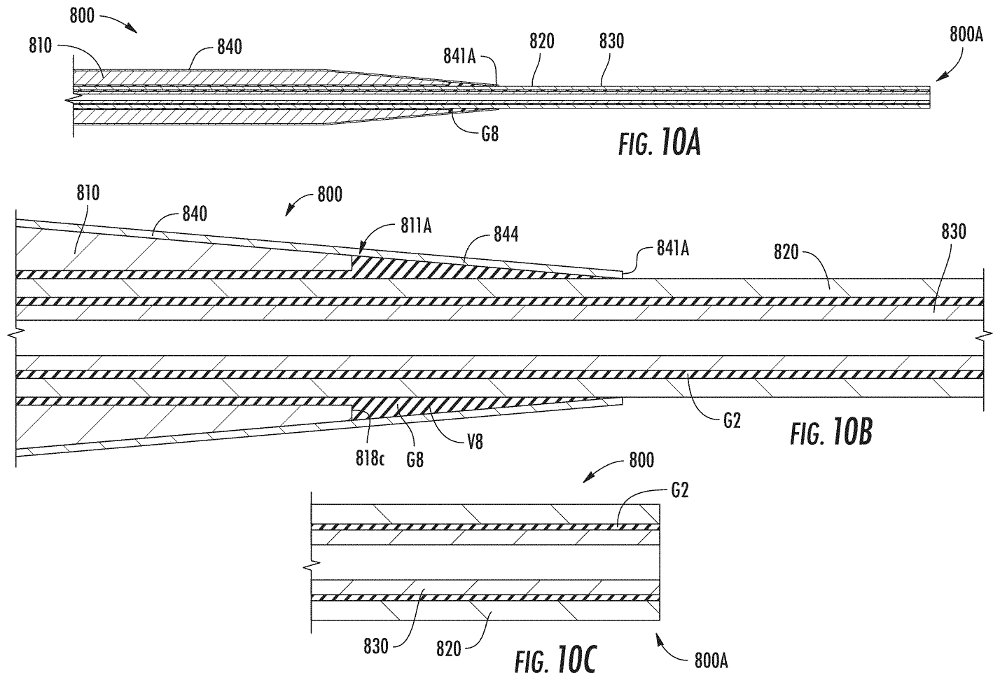

Responsive to the UI feedback, the operator may adjust one or more operational parameters during the fluid delivery. For example, the mass flow rate of the substance exiting the cannula 100 can be increased or decreased. This may be accomplished by adjusting the mass flow rate setting of the pump 82 or a regulator (e.g., restrictor or valve) upstream of the cannula 100. In some cases (e.g., as described below with reference to FIG. 10), the delivery cannula includes a flow control mechanism that can be used to adjust the mass flow rate of delivery.

The operator may adjust the placement of the exit port responsive to the UI feedback. For example, the operator may insert the cannula 100 further into the patient or withdraw the end of the cannula 100 somewhat from the patient. The operator may fully withdraw the cannula 100 from the patient, plan a new target and trajectory, re-insert the cannula 100 to the new target, and re-initiate delivery of the substance to the new target through the cannula 100. This procedure may be used one or more times in order to dispense the substance into different regions of a tumor, for example. Different cannulas or a protective (e.g., retractable) sheath may be used to inhibit spread of tumor cells.

With reference to FIGS. 8A-8F, a cannula system 601 according to embodiments of the present invention is shown therein. The system 601 includes an intrabody surgical (e.g., delivery) cannula 600 (hereinafter, the delivery cannula 600), which can optionally be MRI compatible. The cannula 600 can be coupled to connecting tubing 660, and a luer fitting (e.g., luer lock) 652. The cannula 600 may be used in place of the cannula 100 and the connecting tubing 660 may be used in place of the connecting tubing 84. The cannula 600 may be particularly well-suited for delivering a substance into a patient.

The cannula 600 includes a rigid tubular support sleeve 610, an inner sleeve 620, a transfer tube 630, an outer conformal polymeric sleeve 640, and adhesives G1 and G2. The cannula 600 extends from a proximal end 600B to a distal end 600A.

The rigid tubular support sleeve 610 defines an axially extending central lumen 612. A first or inlet opening 616 on the proximal end of the support sleeve 610 and a second or exit opening 614 on the distal end 611A of the support sleeve 610 each fluidly communicate with the lumen 612. The outer surface 618 of the support sleeve 610 includes a proximal section 618A having a substantially uniform diameter D8. The outer surface 618 also includes a distal section 618B having a tapered or frusto-conical shape that tapers in the axial distal direction. The outer surface 618 further includes a distal end face 618C at the distal end 611A.

According to some embodiments, the support sleeve 610 is formed of a substantially rigid MRI-compatible material. According to some embodiments, the support sleeve 610 is formed of an MR safe material. According to some embodiments, the MRI-compatible material is a ceramic. Suitable ceramics may include Alumina. Other MRI-compatible materials that may be used for the sleeve 610 may include glass or rigid polymers.

The inner sleeve 620 extends through the lumen 612 to a distal end 621A of the inner sleeve. The inner sleeve 620 is secured to the inner surface of the support sleeve 610. According to some embodiments, the inner sleeve 620 is bonded to the inner surface of the support sleeve 610 by a layer of adhesive G1 such as LOCTITE® 4014 adhesive.

The inner sleeve 620 defines an axially extending central lumen 622. A first or inlet opening 626 on the proximal end of the inner sleeve 620 and a second or exit opening 624 on the distal end 621A of the inner sleeve 620 each fluidly communicate with the lumen 622. An extension section 620A of the inner sleeve 620 extends beyond the distal end of the support sleeve 610 and is exposed. The distal end 621A of the inner sleeve 620 defines a distal end face 620B. A proximal extension section 620C of the inner sleeve 620 extends beyond the proximal end of the support sleeve 610 and through the connecting tubing 660 to a delivery pump, for example.

According to some embodiments, the inner sleeve 620 is formed of a substantially rigid MRI-compatible material. According to some embodiments, the MRI-compatible material is fused silica.

A transfer tube 630 extends through the lumen 622 to a distal end 631A of the transfer tube 630. The transfer tube 630 is secured to the inner surface of the inner sleeve 620. According to some embodiments, the transfer tube 630 is bonded to the inner surface of the inner sleeve 620 by a layer of adhesive G2, such as LOCTITE® 4014 adhesive.

The transfer tube 630 defines an axially extending central lumen 632. A first or inlet opening 636 on the proximal end of the transfer tube 630 and a second or exit opening 634 on the distal end 631A of the transfer tube 630 each fluidly communicate with the lumen 632. A distal extension section 630A of the transfer tube 630 extends beyond the distal end 620A of the inner sleeve 620 and is exposed. A proximal extension section 630C (FIG. 8C) of the transfer tube 630 extends beyond the proximal end of the support sleeve 610 and through the connecting tubing 660 to a delivery pump, for example.

According to some embodiments, the transfer tube 630 is formed of a substantially rigid MRI-compatible material. According to some embodiments, the MRI-compatible material is fused silica.

The outer conformal outer polymeric sleeve 640 surrounds and fits tightly about the support sleeve 610. The conformal polymeric sleeve 640 extends from a proximal end proximate the connecting tubing 660 to a distal end 641A (FIG. 8E) at the distal end 621A of the inner sleeve 620. Thus, the conformal polymeric sleeve 640 is coterminous with the inner sleeve 620 at their distal ends.

With reference to FIG. 8F, the conformal polymeric sleeve 640 includes a proximal section 642, a transitional section 644, and a distal section 646. The proximal section 642 surrounds a portion of the support sleeve 610. The distal section 646 surrounds a portion of the inner sleeve 620. The transitional section 644 begins at and extends from the distal end 611A of the support sleeve 610 and over the inner sleeve 620 in the direction toward the distal end 621A of the inner sleeve 620 (i.e., toward the tip of the cannula 600). The transitional section 644 connects the sections 642 and 646 and surrounds a portion of the inner sleeve 620 adjacent the distal end 611A of the support sleeve 610.

The transitional section 644 is frustoconical and tapers inwardly in axial distal direction. In this manner, the transitional section 644 reduces in diameter from the outer diameter of the support sleeve 610 at the end face 614 to the outer diameter of the inner sleeve 620.

An annular void V6 is defined between the outer surface of the inner sleeve 620 and the inner surface of the transitional section 644. The outer diameter of the void V6 is frusto-conical. The void V6 extends from the distal end 611A of the support sleeve 610 to the location or region R6 (FIG. 8F) where the conformal sleeve meets, engages, or conforms to the outer diameter surface of the inner sleeve 620.

The void V6 is at least partially filled with the solid, rigid adhesive G1. As a result, the transitional section 644 and the adhesive G1 therein form a solid ramp from the outer diameter of the inner sleeve 620 to the outer diameter of the support sleeve distal end section 618B. In this way, the step at the support sleeve end face 618C is effectively smoothed of eliminated on the outer diameter of the cannula 600. In some embodiments, the void V6 is fully filled with the solid, rigid adhesive G1.

In some embodiments, the length L12 (FIG. 8F) is in the range of from about 0.1 mm to 5 mm.

The distal section 646 surrounds the remainder of the inner sleeve 620 extending beyond the location R6. The distal section 646 extends from the location R6 to the distal end 641A of the conformal polymeric sleeve 640. The distal section 646 is thus coterminous with the inner sleeve 620 at the distal end 621A. In some embodiments, the polymeric sleeve distal section 646 wraps around the end 621A of the inner sleeve 620, extends radially inward to the transfer tube 630, and covers the inner sleeve end face 620B.

According to some embodiments, the conformal polymeric sleeve 640 is formed of polyethylene terephthalate (PET). According to some limits, the conformal polymeric sleeve 640 is an elastomeric shrinkable sleeve.

The connecting tubing 660 (FIGS. 8A-8D) is coupled to the proximal end of the support sleeve 610 by a tubing adapter 662. The tubing adapter 662 may be bonded to the support sleeve 610 and the connecting tubing 660 by an adhesive G3, such as LOCTITE™ UV 3311 adhesive. The connecting tubing 660 may be formed of any suitable MRI compatible material. According to some embodiments, the connecting tubing 660 is formed of polyvinyl chloride (PVC). According to some embodiments, the connecting tubing 660 is formed of silicone. The tubing adapter 662 may be formed of any suitable MRI compatible material, such as an MRI-compatible polymer.

The luer fitting 652 is coupled to the proximal end of the connecting tubing 660 by a luer adapter 654. The luer adapter 654 may be bonded to the luer fitting 652 and the connecting tubing 660 by an adhesive G4, such as LOCTITE™ UV 3311 adhesive. The luer adapter 654 may be formed of any suitable MRI compatible material, such as an MRI-compatible polymer. The luer fitting 652 may also be bonded to the inner sleeve 620 by one or more the adhesives G5, G6, such as LOCTITE® UV 3311 adhesive and/or LOCTITE® UV 4014 adhesive.

According to some embodiments, the inner diameter D1 of the transfer tube 630 is in the range of from about 10 μm to 1 mm and, in some embodiments, is about 200 μm. According to some embodiments, the outer diameter D2 of the transfer tube 630 is in the range of from about 75 μm to 1.08 mm and, in some embodiments is about 360 μm. According to some embodiments, the length L1 of the exposed section 630A of the transfer tube 630 is in the range of from about 1 mm to 50 mm and, in some embodiments is about 3 mm.

According to some embodiments, the inner diameter D4 of the inner sleeve 620 is in the range of from about 85 μm to 1.1 mm and, in some embodiments, is about 450 μm. According to some embodiments, the outer diameter D5 of the inner sleeve 620 is in the range of from about 150 μm to 1.5 mm and, in some embodiments, is about 673 μm. According to some embodiments, the length L4 of the exposed section 620A of the inner sleeve 620 (i.e., the section of the inner sleeve 620 extending distally beyond the support sleeve end face 618C) is in the range of from about 1 mm to 75 mm and, in some embodiments is about 15 mm.

According to some embodiments, the inner diameter D7 of the support sleeve 610 is in the range of from about 160 μm to 1.55 mm and, in some embodiments, is about 750 μm. According to some embodiments, the outer diameter D8 of the uniform diameter section 618A of the support sleeve 610 is in the range of from about 500 μm to 4 mm and, in some embodiments, is about 1.6 mm. According to some embodiments, the overall length L7 of the support sleeve 610 is in the range of from about 0.5 inch to 20 inches and, in some embodiments, is in the range of from about 10 to 14 inches. According to some embodiments, the length L8 of the tapered section 618B of the support sleeve 610 is in the range of from about 6 to 9 mm.

According to some embodiments, the thickness of the conformal polymeric sleeve 640 is in the range of from about 40 to 60 μm.

As best seen in FIG. 8E, the cannula 600 is a stepped cannula with three co-axially disposed step segments (the outer surfaces of the transfer tube 630, the inner sleeve 620 and the conformal polymeric sleeve 640, respectively) having different outer diameters and separated by the steps or rises. The step formed by the end face 620B is a sharp step. The frustoconical sections 644, 618B form a transitional section T of the cannula 600 having a smooth rise (i.e., not a sharp step).

The cannula 600 may be a unitary, integral structure having no relatively slidably elements.

The cannula 600 may be used in the same manner as described herein with respect to the cannula 100, for example. The luer can be operatively coupled to an infusion pump (e.g., the infusion pump 82) or syringe, which supplies a mass flow of the desired substance or material to be delivered into the patient.

The cannula 600 can provide a number of advantages. The rigid support sleeve 610 prevents or inhibits bending or flex of the large majority of the length of the cannula 600 as the cannula 600 is inserted through the targeting cannula 60 and into the patient (e.g., the brain). By restricting the axial movement of the cannula 600 during insertion, the cannula 600 can reduce or prevent small movements that may disrupt tissue and thereby lead to reflux of the dispensed or infused substance. A ceramic support sleeve 610, in particular, can provide good rigidity while also being MRI-compatible and MM safe. According to some embodiments, the entirety of the cannula 600 is formed of an MRI-compatible, MR safe material or materials.

The conformal polymeric sleeve 640 may beneficially provide a lubricious surface over the support sleeve 610 to reduce shear force on the brain or other tissue during insertion. The conformal polymeric sleeve 640 can enhance the safety of the cannula 600 by capturing the support sleeve 610 or pieces thereof if the support sleeve is accidentally broken in situ.

The step at the end face 620B can serve to reduce or prevent reflux of the delivered substance. The provision of an exposed transfer tube section 630A having the aforedescribed length L1 and inner diameter D1 has also been found to provide beneficial reflux resistance performance.

The tapered transition between the outer diameter D5 of the inner sleeve 620 and the outer diameter D7 of the inner sleeve 620 can provide the reflux control of the small diameter inner sleeve 620 along with a support sleeve 610 having a geometry providing satisfactory rigidity and size for cooperation with the targeting cannula 60 or an adapter.

The protective connecting tubing 660 can serve to protect the transfer tube 630 while also permitting convenient routing the connecting tubing 660 to the delivery pump. According to some embodiments, the length of the tubing 660 is in the range of from about 6 to 12 feet.

According to some embodiments, the delivered substance is delivered to a patient's brain through the exit opening 634 at a delivery rate in the range of from about 1 to 3 μL/minute.

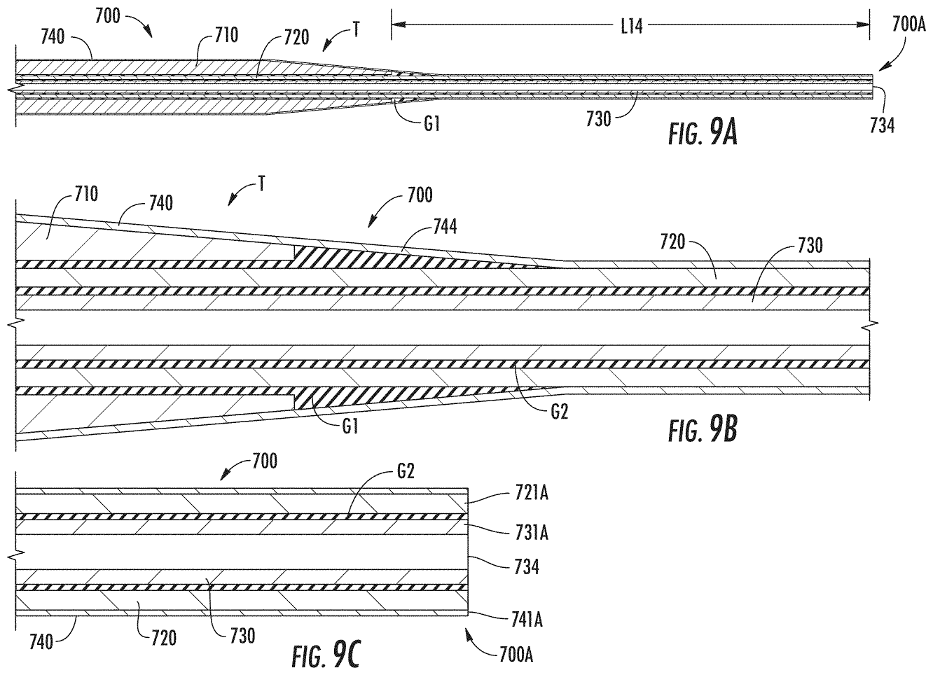

With reference to FIGS. 9A-9C, a cannula 700 according to further embodiments is shown therein. The cannula 700 is constructed in the same manner as the cannula 600, except as discussed below. The cannula 700 can be used in place of and in the same manner described for the cannula 600 (e.g., with the connecting tubing 660, and a luer fitting (e.g., luer lock) 652). The cannula 700 may be particularly well-suited for delivering a substance to a patient.

The cannula 700 includes a rigid tubular support sleeve 710, an inner sleeve 720, a transfer tube 730, a conformal polymeric sleeve 740, an adhesive G1, and an adhesive G2 constructed and assembled in the same manner as the support sleeve 610, the inner sleeve 620, the transfer tube 630, the conformal polymeric sleeve 640, the adhesive G1, and the adhesive G2, respectively, of the cannula 600 except as discussed below. The cannula 700 extends from a proximal end to a distal end 700A. The cannula 700 includes a conformal sleeve transitional section 744 and a transitional section T corresponding to the conformal sleeve transitional section 644 and the transitional section T of the cannula 600.

The cannula 700 differs from the cannula 600 in that the distal end 721A (FIG. 9C) of the inner sleeve 720 is substantially coterminous or coincident with the distal end 731A of the transfer tube 730.

As a result, the cannula 700 does not include a step corresponding to the step at the distal end 621A of the inner sleeve 620. The cannula 700 includes only the smoothly tapered rise at the transitional section T. The cannula 700 has a constant outer diameter after the rise at the transitional section T to the tip.

In the cannula 700, the conformal polymeric sleeve 740 terminates at the distal end 721A of the inner sleeve 620 as described for the cannula 600 (i.e., the conformal sleeve distal end 741A (FIG. 9C) is coterminous with the distal end 721A and the cannula distal end 700A).

In some embodiments, the length L14 (FIG. 9A) of the transfer tube 730 extending beyond the support sleeve 710 is in the range of from about 3 mm to 8 cm.

The cannula 700 may be particularly well-suited for delivering stem cells to the brain tissue of a patient. According to method embodiments of the invention, stem cells are delivered or injected through the cannula 700 into a patient as described herein. In some embodiments, the stem cells are injected into patient brain tissue through the cannula 700. The stem cells may be suspended in a liquid composition or suspension that is delivered through the cannula 700.

In some embodiments, the stems cells are delivered to the patient tissue in the following manner. The cannula 700 is inserted into the patient anatomy as described herein to position the distal end 700A in or proximate a target region. The cannula 700 is then pulled back or withdrawn from the patient a small or prescribed distance such that the distal end 700A remains in the patient in or proximate the target region, and a gap or channel is thereby formed in the patient tissue where the distal end section of the cannula was formerly located. The stem cells are then dispensed into the patient, including into the formed channel, through the cannula 700 and the distal end opening 734. In some embodiments, the cannula 700 is then again pulled back or withdrawn from the patient a small or prescribed distance such that the distal end 700A remains in the patient in or proximate the target region, and a further gap or channel is thereby formed in the patient tissue where the distal end section of the cannula was formerly located. The stem cells are then dispensed into the patient, including into the second formed channel, through the cannula 700 and the distal end opening 734. This procedure (withdraw the cannula incrementally and inject stem cells) may be repeated any suitable or desired number of times.

In some embodiments, the stem cells are implanted, dispensed or injected into the gap or channel with relatively little or no supplemental pressure applied to the stem cells. Because the stem cells are dispensed into the gap or channel, in some embodiments it is not necessary to pressurize the delivered liquid in order to force the liquid into the patient tissue as desired.

With reference to FIGS. 10A-10C, a cannula 800 according to further embodiments is shown therein. The cannula 800 is constructed in the same manner as the cannula 700, except as discussed below. The cannula 800 can be used in place of and in the same manner described for the cannula 600 (e.g., with the connecting tubing 660, and a luer fitting (e.g., luer lock) 652).

The cannula 800 includes a rigid tubular support sleeve 810, an inner sleeve 820, a transfer tube 830, a conformal polymeric sleeve 840, an adhesive G8, and an adhesive G2 constructed and assembled in the same manner as the support sleeve 710, the inner sleeve 720, the transfer tube 730, the conformal polymeric sleeve 740, the adhesive G1, and the adhesive G2, respectively, of the cannula 700, except as discussed below. The cannula 800 extends from a proximal end to a distal end 800A.

The cannula 800 differs from the cannula 700 in that the distal end 841A of the conformal polymeric sleeve 840 is proximate the distal end 811A of the rigid support sleeve 810 between the distal end 811A and the distal end 821A of the inner sleeve 820. The conformal polymeric sleeve 840 extends over and surrounds the inner sleeve 830.

The conformal polymeric sleeve 840 includes a transitional section 844 corresponding to the transitional section 644 (FIG. 8F) and that is frustoconical and tapers inwardly in the axial distal direction. The transitional section 844 reduces in diameter from the outer diameter of the support sleeve 810 at the end face 814 to the outer diameter of the inner sleeve 820.

An annular void V8 (corresponding to the annular void V6 of FIG. 8F) is defined between the outer surface of the inner sleeve 820 and the inner surface of the transitional section 844. The void V8 is at least partially filled with the solid, rigid adhesive G8. As a result, the transitional section 844 and the adhesive G8 therein form a solid ramp from the outer diameter of the inner sleeve 820 to the outer diameter of the support sleeve distal end section. In this way, the step at the support sleeve end face 818C is effectively smoothed or eliminated on the outer diameter of the cannula 800. In some embodiments, the void V8 is fully filled with the solid, rigid adhesive G8.

In alternative embodiments (not shown in the drawings), the cannula 800 is constructed such that the distal end 841A of the conformal polymeric sleeve 840 is located at the distal end 811A of the rigid support sleeve 810. In this case, no void V8 is formed and the adhesive G8 does not extend beyond the support sleeve 810. In some embodiments, the conformal polymeric sleeve 840 covers the distal end face of the support sleeve 810 down to the outer diameter of the inner sleeve 820 to smooth the transition from the outer diameter of the support sleeve 810.

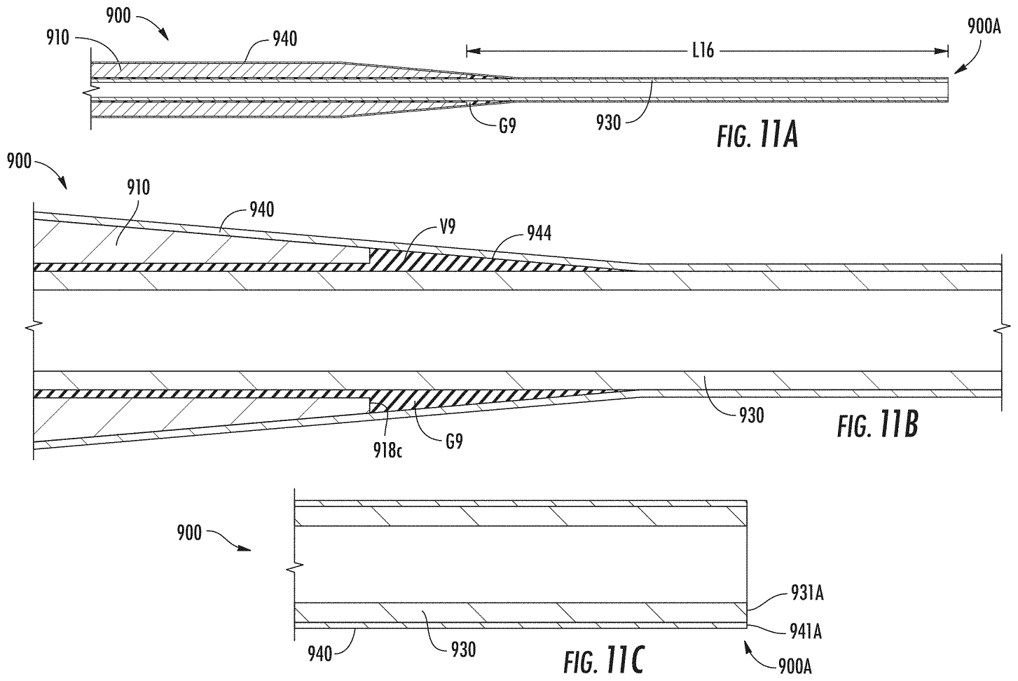

With reference to FIGS. 11A-11C, a cannula 900 according to further embodiments is shown therein. The cannula 900 is constructed in the same manner as the cannula 600, except as discussed below. The cannula 900 can be used in place of and in the same manner described for the cannula 600 (e.g., with the connecting tubing 660, and a luer fitting (e.g., luer lock) 652).

The cannula 900 includes a rigid tubular support sleeve 910, a transfer tube 930, a conformal polymeric sleeve 940, and an adhesive G9 constructed and assembled in the same manner as the support sleeve 610, the transfer tube 630, the conformal polymeric sleeve 640, and the adhesive G2, respectively, of the cannula 600 except as discussed below. The cannula 900 extends from a proximal end to a distal end 900A.

The cannula 900 differs from the cannula 600 in that the cannula 900 does not include a component corresponding to the inner sleeve 620. Instead, the outer diameter of the transfer tube 930 is bonded directly to the inner diameter of the support sleeve 910 by the adhesive G2.

The conformal polymeric sleeve 940 extends over and surrounds the transfer tube 930. The distal end 941A of the conformal polymeric sleeve 940 is substantially coterminous or coincident with the distal end 931A of the transfer tube 930.

The conformal polymeric sleeve 940 includes a transitional section 944 corresponding to the transitional section 644 (FIG. 8E) and that is frustoconical and tapers inwardly in axial distal direction. The transitional section 944 reduces in diameter from the outer diameter of the support sleeve 910 at the end face 914 to the outer diameter of the transfer tube 930.

An annular void V9 (corresponding to the annular void V6 of FIG. 8F) is defined between the outer surface of the transfer tube 930 and the inner surface of the transitional section 944. The void V9 is at least partially filled with the solid, rigid adhesive G9. As a result, the transitional section 944 and the adhesive G9 therein form a solid ramp from the outer diameter of the transfer tube 930 to the outer diameter of the support sleeve distal end section 918B. In this way, the step at the support sleeve end face 918C is effectively smoothed or eliminated on the outer diameter of the cannula 900. In some embodiments, the void V9 is fully filled with the solid, rigid adhesive G9.

In some embodiments, the length L16 (FIG. 11A) of the transfer tube 930 extending beyond the support sleeve 910 is in the range of from about 3 mm to 8 cm.

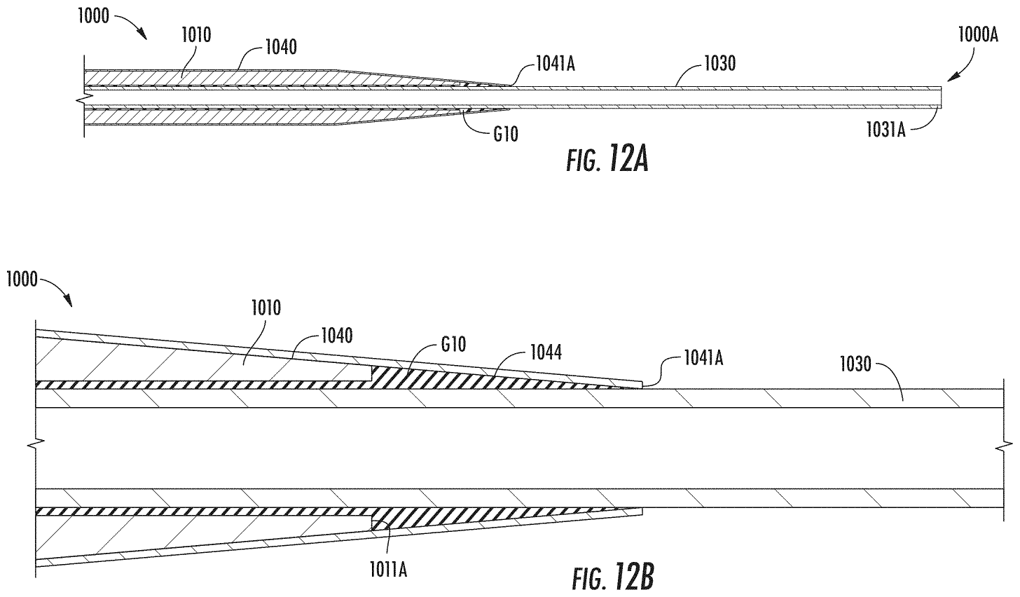

With reference to FIGS. 12A-12B, a cannula 1000 according to further embodiments is shown therein. The cannula 1000 is constructed in the same manner as the cannula 900, except as discussed below. The cannula 1000 can be used in place of and in the same manner described for the cannula 600 (e.g., with the connecting tubing 660, and a luer fitting (e.g., luer lock) 652).

The cannula 1000 includes a rigid tubular support sleeve 1010, a transfer tube 1030, a conformal polymeric sleeve 1040, and an adhesive G10 constructed and assembled in the same manner as the support sleeve 910, the transfer tube 930, the conformal polymeric sleeve 940, and the adhesive G9, respectively, of the cannula 900 except as discussed below. The cannula 1000 extends from a proximal end to a distal end 1000A. The cannula 1000 includes a conformal sleeve transitional section 1044 and a transitional section T corresponding to the conformal sleeve transitional section 944 and the transitional section T of the cannula 900.

The cannula 1000 differs from the cannula 900 in that the distal end 1041A of the conformal polymeric sleeve 1040 is located at or proximate the distal end 1011A of the rigid support sleeve 1010. In some embodiments, the polymeric sleeve 1040 covers the distal end face of the support sleeve 1010 down to the outer diameter of the transfer tube 1030 to smooth the transition from the outer diameter of the support sleeve 1010.

The cannula 1000 differs from the cannula 900 in that the distal end 1041A of the conformal polymeric sleeve 1040 is proximate the distal end 1011A of the rigid support sleeve 1010 between the distal end 1011A and the distal end 1031A of the transfer tube 1030. The conformal polymeric sleeve 1040 extends over and surrounds the transfer tube 1030.

In alternative embodiments (not shown in the drawings), the cannula 1000 is constructed such that the distal end 1041A of the conformal polymeric sleeve 1040 is located at the distal end 1011A of the rigid support sleeve 1010. In some embodiments, the polymeric sleeve 1040 covers the distal end face of the support sleeve 1010 down to the outer diameter of the transfer tube 1030 to smooth the transition from the outer diameter of the support sleeve 1010.

The cannulas 800, 900, 1000 may also be particularly well-suited for delivering stem cells to the brain tissue of a patient. In particular, the cannulas 800, 900, 1000 may be well-suited for implanting stem cells into the brain tissue of a patient. The cannulas 800, 900, 1000 may be used in the same manner as the cannula 700 to dispense stem cells into a patient.

As discussed herein, insertion of the surgical cannula 100 (or any other surgical, e.g., delivery, cannula) can be tracked in near real time by reference to a void in the patient tissue caused by the cannula 100 and reflected in the MR image. In some embodiments, one or more MRI-visible fiducial markers may be provided on the surgical cannula 100, MR scanned and processed, and displayed on the UI. In some embodiments, the surgical cannula 100 may itself be formed of an MRI-visible material, MR scanned and processed, and displayed on the UI.

According to some embodiments, the surgical cannula may include an embedded intrabody MRI antenna that is configured to pick-up MRI signals in local tissue during an MM procedure. The MRI antenna can be configured to reside on a distal end portion of the surgical cannula. In some embodiments, the antenna has a focal length or signal-receiving length of between about 1-5 cm, and typically is configured to have a viewing length to receive MRI signals from local tissue of between about 1-2.5 cm. The MRI antenna can be formed as comprising a coaxial and/or triaxial antenna. However, other antenna configurations can be used, such as, for example, a whip antenna, a coil antenna, a loopless antenna, and/or a looped antenna. See, e.g., U.S. Pat. Nos. 5,699,801; 5,928,145; 6,263,229; 6,606,513; 6,628,980; 6,284,971; 6,675,033; and 6,701,176, the contents of which are hereby incorporated by reference as if recited in full herein. See also U.S. Patent Application Publication Nos. 2003/0050557; 2004/0046557; and 2003/0028095, the contents of which are also hereby incorporated by reference as if recited in full herein.

Surgical cannulas 600, 700, 800, 900, 1000 as described herein may be used with a stereotactic frame or without a stereotactic frame.

While the surgical cannulas 600, 700, 800, 900, 1000 have been identified herein as delivery cannulas and methods for delivering a substance to a patient have been described, in accordance with some embodiments of the invention, the surgical cannulas and methods can be used to withdraw a substance (e.g., spinal fluid) from a patient. Thus, it will be appreciated that surgical cannulas and methods as disclosed herein can be used to transfer a substance into and/or from a patient.

While the surgical cannulas 600, 700, 800, 900, 1000 have been described herein with reference to MM-guided insertion and infusion procedures, in some embodiments the cannulas can be used in procedures without Mill guidance, such as CT-guided systems and may use other materials than described above.

While the surgical cannulas 600, 700, 800, 900, 1000 have been described in use with a trajectory guide 50b, the cannulas may be used with other types of trajectory guidance or stereotactic frames or without a stereotactic frame or trajectory guide.

The surgical cannulas 600, 700, 800, 900, 1000 as depicted in FIGS. 8A-12B may typically be employed for acute treatments. However, the systems, cannulas, methods and procedures described herein may likewise be used a chronic delivery cannula or catheter. For example, the delivery cannulas 600, 700, 800, 900, 1000 may be installed on a patient for chronic delivery as described in PCT Published Patent Application No. WO 2011/130107 A2 (Attorney Docket No. 9450-75WO).

According to some embodiments, the substance delivered via the delivery cannula includes radioactive objects such as radioactive seeds. In this event, the delivery cannula may include a suitable radiation shield or shielding material in order to reduce or prevent the exposure of tissue outside the target region to radiation from the radioactive objects.

The system 10 may also include a decoupling/tuning circuit that allows the system to cooperate with an Mill scanner 20 and filters and the like. See, e.g., U.S. Pat. Nos. 6,701,176; 6,904,307 and U.S. Patent Application Publication No. 2003/0050557, the contents of which are hereby incorporated by reference as if recited in full herein.

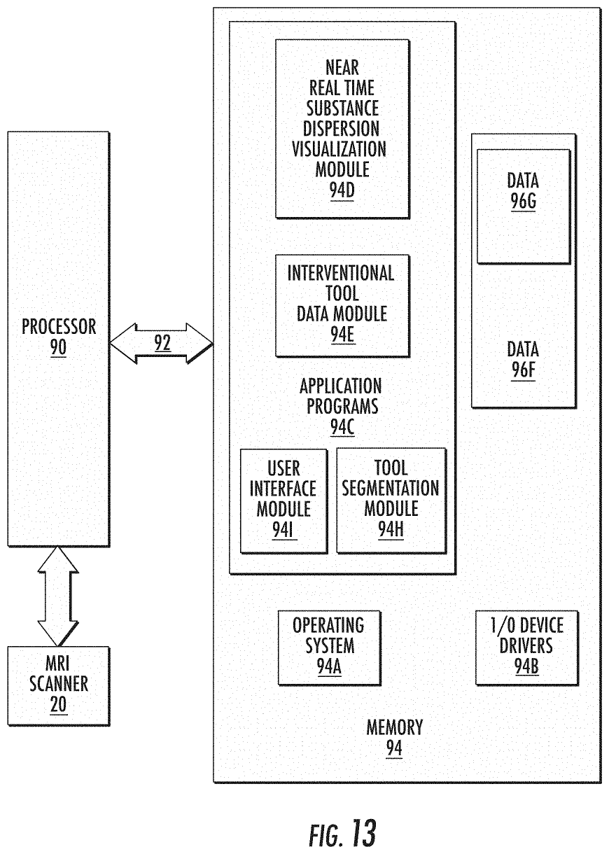

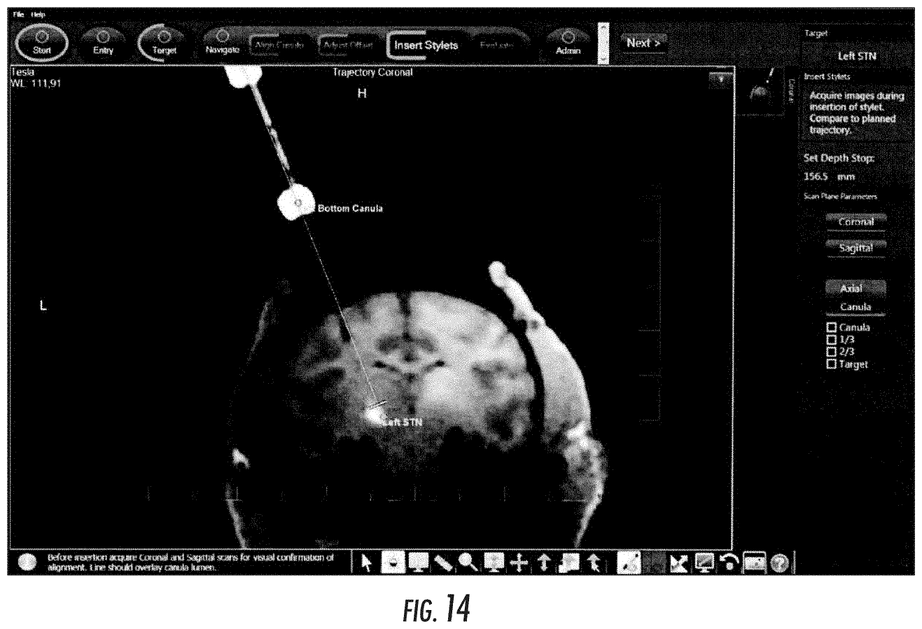

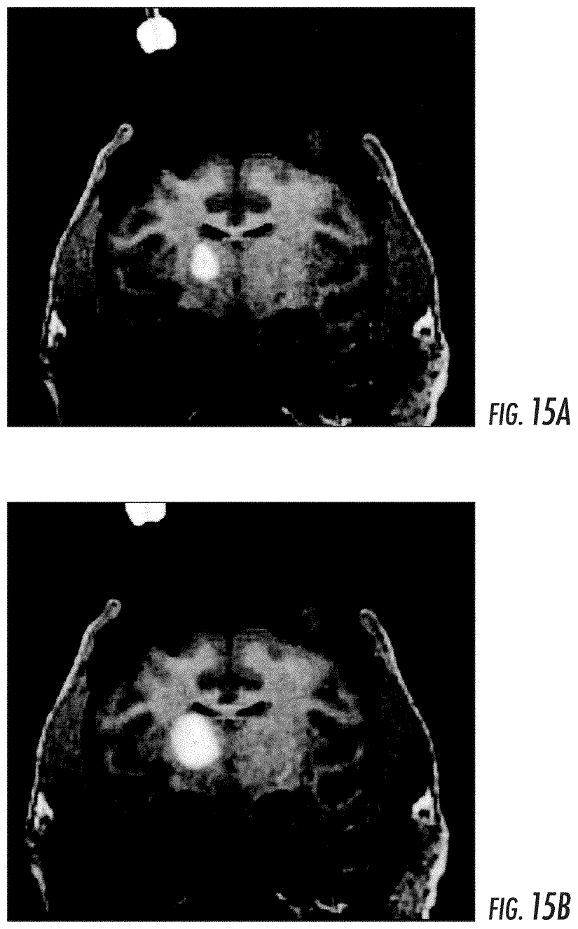

The system 10 can include circuits and/modules that can comprise computer program code used to automatically or semi-automatically carry out operations to generate visualizations and provide output to a user to facilitate MM-guided diagnostic and therapy procedures. FIG. 13 is a schematic illustration of a circuit or data processing system that can be used with the system 10. The circuits and/or data processing systems may be incorporated in one or more digital signal processors in any suitable device or devices. The processor 90 communicates with an MRI scanner 20 and with memory 94 via an address/data bus 92. The processor 90 can be any commercially available or custom microprocessor. The memory 94 is representative of the overall hierarchy of memory devices containing the software and data used to implement the functionality of the data processing system. The memory 94 can include, but is not limited to, the following types of devices: cache, ROM, PROM, EPROM, EEPROM, flash memory, SRAM, and DRAM.

The memory 94 may include several categories of software and data used in the data processing system: the operating system 94A; the application programs 94C; the input/output (I/O) device drivers 94B; and data 94F. The data 94F can also include predefined characteristics of different surgical tools and patient image data 94G. The application programs 94C can include a Near Real-Time Substance Dispersion Visualization Module 94D, Interventional Tool Data Module 94E, a Tool Segmentation Module 9411 (such as segmentation modules for a targeting cannula, a trajectory guide frame and/or base, and a delivery cannula), and a workflow group User Interface Module 94I (that facilitates user actions and provides guidance to obtain a desired trajectory or a desired drug dispersion pattern, such as physical adjustments to achieve same).

As will be appreciated by those of skill in the art, the operating systems 94A may be any operating system suitable for use with a data processing system, such as OS/2, AIX, DOS, OS/390 or System390 from International Business Machines Corporation, Armonk, N.Y., Windows CE, Windows NT, Windows95, Windows98, Windows2000 or other Windows versions from Microsoft Corporation, Redmond, Wash., Unix or Linux or FreeBSD, Palm OS from Palm, Inc., Mac OS from Apple Computer, LabView, or proprietary operating systems. The I/O device drivers 94C typically include software routines accessed through the operating system 94A by the application programs 94C to communicate with devices such as I/O data port(s), data storage 94F and certain memory 94 components. The application programs 94C are illustrative of the programs that implement the various features of the data processing system and can include at least one application, which supports operations according to embodiments of the present invention. Finally, the data 94F represents the static and dynamic data used by the application programs 94C, the operating system 94A, the I/O device drivers 94C, and other software programs that may reside in the memory 94.

While the present invention is illustrated, for example, with reference to the Modules 94C, 94D, 94E, 94H, 94I being application programs in FIG. 13, as will be appreciated by those of skill in the art, other configurations may also be utilized while still benefiting from the teachings of the present invention. For example, the Modules 94C, 94D, 94E, 94H, 94I and/or may also be incorporated into the operating system 94A, the I/O device drivers 94B or other such logical division of the data processing system. Thus, the present invention should not be construed as limited to the configuration of FIG. 13, which is intended to encompass any configuration capable of carrying out the operations described herein. Further, one or more of modules, i.e., Modules 94C, 94D, 94E, 94H, 94I can communicate with or be incorporated totally or partially in other components, such as a workstation, an MRI scanner, an interface device. Typically, the workstation 30 will include the modules 94C, 94D, 94E, 94H, 94I and the MR scanner with include a module that communicates with the workstation 30 and can push image data thereto.

The I/O data port can be used to transfer information between the data processing system, the circuit 30c or workstation 30, the MRI scanner 20, and another computer system or a network (e.g., the Internet) or to other devices controlled by or in communication with the processor. These components may be conventional components such as those used in many conventional data processing systems, which may be configured in accordance with the present invention to operate as described herein.

It is noted that any one or more aspects or features described with respect to one embodiment, may be incorporated in a different embodiment although not specifically described relative thereto. That is, all embodiments and/or features of any embodiment can be combined in any way and/or combination. Applicant reserves the right to change any originally filed claim or file any new claim accordingly, including the right to be able to amend any originally filed claim to depend from and/or incorporate any feature of any other claim although not originally claimed in that manner. These and other objects and/or aspects of the present invention are explained in detail in the specification set forth below.

Other systems, methods, and/or computer program products according to embodiments of the invention will be or become apparent to one with skill in the art upon review of the following drawings and detailed description. It is intended that all such additional systems, methods, and/or computer program products be included within this description, be within the scope of the present invention, and be protected by the accompanying claims.

In the drawings and specification, there have been disclosed embodiments of the invention and, although specific terms are employed, they are used in a generic and descriptive sense only and not for purposes of limitation, the scope of the invention being set forth in the following claims. Thus, the foregoing is illustrative of the present invention and is not to be construed as limiting thereof. More particularly, the workflow steps may be carried out in a different manner, in a different order and/or with other workflow steps or may omit some or replace some workflow steps with other steps. Although a few exemplary embodiments of this invention have been described, those skilled in the art will readily appreciate that many modifications are possible in the exemplary embodiments without materially departing from the novel teachings and advantages of this invention. Accordingly, all such modifications are intended to be included within the scope of this invention as defined in the claims. In the claims, means-plus-function clauses, where used, are intended to cover the structures described herein as performing the recited function and not only structural equivalents but also equivalent structures. Therefore, it is to be understood that the foregoing is illustrative of the present invention and is not to be construed as limited to the specific embodiments disclosed, and that modifications to the disclosed embodiments, as well as other embodiments, are intended to be included within the scope of the appended claims. The invention is defined by the following claims, with equivalents of the claims to be included therein.

1. A cannula for transferring a substance to and/or from a patient, the cannula comprising:
a rigid, tubular support sleeve defining a support sleeve lumen extending from a first opening at a proximal end of the support sleeve to a second opening at a distal end of the support sleeve, wherein the support sleeve comprises a rigid, MRI-compatible material;
a transfer tube disposed in the support sleeve lumen and extending beyond the distal end of the support sleeve to a distal end of the transfer tube, the transfer tube defining a transfer tube lumen terminating at an opening at the distal end of the transfer tube; and
a conformal polymeric sleeve surrounding at least a portion of the support sleeve and at least a portion of the transfer tube;
wherein:
the conformal polymeric sleeve includes a transitional section extending from the distal end of the support sleeve and over the transfer tube in a direction toward the distal end of the transfer tube; and
the transitional section tapers inwardly in the direction toward the distal end of the transfer tube.

2. The cannula of claim 1 wherein the transitional section is frustoconical.

3. The cannula of claim 1 wherein the transitional section extends from the distal end of the support sleeve toward the distal end of the transfer tube an axial distance in the range of from about 0.1 mm to 5 mm.

4. The cannula of claim 1 wherein the support sleeve comprises a ceramic material.

5. The cannula of claim 1 wherein the conformal polymeric sleeve is a polymeric shrink tubing.

6. The cannula of claim 1 wherein the transfer tube is formed of fused silica.

7. The cannula of claim 1 wherein the transfer tube is adhered by adhesive to an inner surface of the support sleeve.

8. The cannula of claim 7 wherein:
the conformal polymeric sleeve defines an annular void between an outer surface of the transfer tube and the transitional section of the conformal polymeric sleeve adjacent the distal end of the support sleeve; and
the cannula includes an adhesive at least partially filling the annular void.

9. The cannula of claim 7 wherein the conformal polymeric sleeve extends to the distal end of the transfer tube.

10. The cannula of claim 7 wherein the conformal polymeric sleeve has a distal end located axially between the distal end of the support sleeve and the distal end of the transfer tube.

11. The cannula of claim 1 including an inner sleeve disposed in the support sleeve lumen and extending beyond the distal end of the support sleeve to a distal end of the inner sleeve, the inner sleeve defining an inner sleeve lumen, and wherein:
the transfer tube is disposed in the inner sleeve lumen and extends to or beyond the distal end of the inner sleeve to the distal end of the transfer tube;
the conformal polymeric sleeve surrounds at least a portion of the inner sleeve;
the transitional section extends from the distal end of the support sleeve and over the inner sleeve in a direction toward the distal end of the inner sleeve; and
the transitional section tapers inwardly in the direction toward the distal end of the inner sleeve.

12. The cannula of claim 11 wherein:
the conformal polymeric sleeve defines an annular void between an outer surface of the inner sleeve and the transitional section of the conformal polymeric sleeve adjacent the distal end of the support sleeve; and
the cannula includes an adhesive at least partially filling the annular void.

13. The cannula of claim 11 wherein the conformal polymeric sleeve extends to the distal end of the inner sleeve.

14. The cannula of claim 13 wherein the conformal polymeric sleeve covers a terminal end face of the inner sleeve at the distal end of the inner sleeve.

15. The cannula of claim 13 wherein the transfer tube extends axially beyond the inner sleeve in the direction of the distal end of the transfer tube.

16. The cannula of claim 11 wherein the distal end of the inner sleeve is coterminous with the distal end of the transfer tube.

17. The cannula of claim 16 wherein the conformal polymeric sleeve extends to the distal end of the inner sleeve.

18. The cannula of claim 16 wherein the conformal polymeric sleeve has a distal end located axially between the distal end of the support sleeve and the distal end of the inner sleeve.

19. The cannula of claim 11 wherein:
the transfer tube extends beyond the distal end of the inner sleeve;
an exterior surface of the cannula has at least first, second and third co-axially disposed segments, an outer diameter of the second segment being greater than an outer diameter of the first segment, and an outer diameter of the third segment being greater than the outer diameter of the second segment;
the second segment extends between and adjoins each of the first and third segments;
the first segment extends from the distal end of the transfer tube to the distal end of the inner sleeve;
the second segment extends from the distal end of the inner sleeve to a distal end of the transitional section of the conformal polymeric sleeve;
the third segment extends from a proximal end of the transitional section toward the proximal end of the support sleeve;
a terminal end face of the inner sleeve forms a sharp step about the transfer tube between the first segment and the second segment, the terminal end face of the inner sleeve projecting radially outwardly beyond the outer diameter of the first segment; and
the transitional section defines a second step between the second segment and the third segment, the second step having a smooth rise from the outer diameter of the second segment to the outer diameter of the third segment.

20. The cannula of claim 11 wherein the inner sleeve is adhered with adhesive to an inner surface of the tubular support sleeve.

21. The cannula of claim 20 wherein the inner sleeve is adhered with adhesive to an outer surface of the transfer tube.

22. A method of transferring a substance to and/or from a patient, the method comprising:
providing a cannula including:
a rigid, tubular support sleeve defining a support sleeve lumen extending from a first opening at a proximal end of the support sleeve to a second opening at a distal end of the support sleeve, wherein the support sleeve comprises a rigid, MM-compatible material;
a transfer tube disposed in the support sleeve lumen and extending beyond the distal end of the support sleeve to a distal end of the transfer tube, the transfer tube defining a transfer tube lumen terminating at an opening at the distal end of the transfer tube; and
a conformal polymeric sleeve surrounding at least a portion of the support sleeve and at least a portion of the transfer tube;
wherein:
the conformal polymeric sleeve includes a transitional section extending from the distal end of the support sleeve and over the transfer tube in a direction toward the distal end of the transfer tube; and
the transitional section tapers inwardly in the direction toward the distal end of the transfer tube;
inserting the cannula into a selected region in the patient; and
transferring the substance to or from the selected region through the transfer tube lumen.

23. The method of claim 22 including:
partially withdrawing the cannula from a patient tissue in the selected region, thereby forming a channel in the patient tissue; and
delivering stem cells through the cannula into the channel.

24. The method of claim 23 wherein the selected region is the brain.

25.-42. (canceled)
